(19) United States
(12) Patent Application Publication
(10) Pub. No.: US 2008/0221105 A1
(43) Pub. Date: Sep. 11, 2008

(54) SOLUBLE EPOXIDE HYDROLASE INHIBITORS FOR TREATMENT OF METABOLIC SYNDROME AND RELATED DISORDERS

(75) Inventor: Heather Kay Webb Hsu, San Francisco, CA (US)

Correspondence Address: FOLEY & LARDNER LLP, 975 PAGE MILL ROAD, PALO ALTO, CA, 94304, US

(73) Assignee: Arete Therapeutics, Inc.

(21) Appl. No.: 12/021,247

(22) Filed: Jan. 28, 2008

Related U.S. Application Data
(60) Provisional application No. 60/887,124, filed on Jan. 29, 2007.

(51) Int. Cl. A61K 31/4468; A61P 3/00; A61K 31/5375

(52) U.S. Cl. 514/237.5; 514/329

(57) ABSTRACT

Compounds, compositions, and methods for inhibiting the onset of metabolic syndrome and treating related disorders in a subject in need of such therapy are disclosed.

### CROSS-REFERENCE TO RELATED APPLICATIONS

This application claims the benefit under 35 U.S.C. §119(e) of U.S. Provisional Patent Application No. 60/887,124 filed on Jan. 29, 2007, which is hereby incorporated by reference in its entirety.

### FIELD OF THE INVENTION

The present invention generally relates to compounds and methods useful for preventing or inhibiting the onset of metabolic syndrome and for treating conditions associated with metabolic syndrome.

### BACKGROUND

Metabolic syndrome is a disorder characterized by a number of health problems including obesity, high blood pressure, abnormal lipid levels and high blood sugar. Metabolic syndrome has other names such as metabolic syndrome X, cardiometabolic syndrome, insulin resistance syndrome, and diabesity. This syndrome has been estimated to be present in as much as 20% of the current population in the United States. Left untreated, metabolic syndrome represents an increased risk of heart attack, stroke, peripheral vascular disease and type II diabetes (non-insulin dependent diabetes mellitus (NIDDM) risk.

Metabolic syndrome is associated with numerous risk factors including those factors brought on by genetic predisposition as well as those that result from external acquired factors, such as excess body fat, poor diet, and physical inactivity. Insulin resistance, in particular, is associated with genetic predisposition. Acquired factors, such as excess body fat, particularly in the abdominal area, and physical inactivity, can elicit insulin resistance and metabolic syndrome in people genetically predisposed to this condition. The biologic mechanisms at the molecular level between insulin resistance and metabolic risk factors are not fully elucidated and appear to be complex.

Metabolic syndrome is currently treated by addressing the external acquired factors that can contribute to the syndrome. Patients with metabolic syndrome are encouraged to adopt healthier lifestyles by increasing physical activity, reducing their intake of fat and cholesterol, and not smoking. If lifestyle changes are not successful, then prescriptions for the individual components of high blood pressure, high cholesterol and diabetes can be applied. Unfortunately, these individual treatments may serve to exacerbate other conditions present in the patient. For example, insulin sensitizers can cause weight gain thus increasing one of the risk factor elements.

Presently, there is no drug known to have a positive impact on multiple conditions associated with metabolic syndrome. Thus, a need exists for effective methods for treating or inhibiting the onset of metabolic syndrome and the numerous conditions associated with the disorder.

### SUMMARY OF THE INVENTION

This invention provides soluble epoxide hydrolase (sEH) inhibitor compounds and compositions that are useful in inhibiting the onset of metabolic syndrome and in treating multiple conditions associated with metabolic syndrome, such as two or more of incipient diabetes, glucose intolerance, obesity, hypertension, high blood pressure, elevated serum cholesterol, reduced high-density lipoproteins and elevated triglyceride levels.

In one aspect, the invention provides a method for inhibiting the onset of metabolic syndrome in a subject predisposed thereto by administering to the subject an effective amount of a sEH inhibitor.

Another aspect provides a method for treating one or more conditions, or, preferably, two or more conditions, or in another aspect, three or more conditions associated with metabolic syndrome in a subject where the conditions are selected from incipient diabetes, obesity, glucose intolerance, hypertension, high blood pressure, elevated serum cholesterol, reduced high-density lipoproteins, and elevated triglycerides. This method comprises administering to the subject an amount of a sEH inhibitor effective to treat the condition or conditions manifested in the subject.

Yet another aspect provides a method of treating a metabolic condition in a subject, comprising administering to the subject an effective amount of a sEH inhibitor. The metabolic condition is selected from the group consisting of conditions comprising obesity, glucose intolerance, incipient diabetes, hypertension, high blood pressure, elevated serum cholesterol, reduced high-density lipoproteins, and elevated triglycerides, and combinations thereof.

The methods described herein preferably include the administration of an effective amount of a sEH inhibitor of Formula (I), Formula (II), or Formula II(a), or pharmaceutically acceptable salts thereof.

Accordingly, provided herein are sEH inhibitors of Formula (I) or a pharmaceutically acceptable salt thereof:

R¹NHC(═Q)NHR² (I)

wherein:

Q is selected from the group consisting of O and S; and

R¹ and R² are independently selected from the group consisting of substituted alkyl, aryl, substituted aryl, heteroaryl, substituted heteroraryl, cycloalkyl, substituted cycloalkyl, heterocycloalkyl, and substituted heterocycloalkyl.

Also provided are sEH inhibitors of Formula (II) or a pharmaceutically acceptable salt thereof:

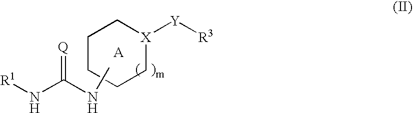

wherein:
Q is selected from the group consisting of O and S;
R¹ is selected from the group consisting of aryl, substituted aryl, heteroaryl, substituted heteroraryl, cycloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂;
R³ is selected from the group consisting of alkyl, substituted alkyl, or heterocycloalkyl; and
m is selected from the group consisting of zero, 1, and 2.

Also provided are sEH inhibitors of Formula (IIa) or a pharmaceutically acceptable salt thereof:

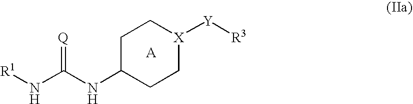

wherein:
Q is selected from the group consisting of O and S;
R¹ is selected from the group consisting of aryl, substituted aryl, heteroaryl, substituted heteroraryl, cycloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂; and
R³ is selected from the group consisting of alkyl, substituted alkyl, or heterocycloalkyl.

In a particular aspect of this invention, the compound to be administered is selected from the group consisting of:
1-[1-(methylsulfonyl)piperidin-4-yl]-3-(adamant-1-yl)urea;
1-[1-(acetyl)piperidin-4-yl]-3-(adamant-1-yl)urea;
1-[1-(acetyl)piperidin-4-yl]-3-(4-trifluoromethylphenyl)urea;
1-[1-(methylsulfonyl)piperidin-4-yl]-3-(4-trifluoromethylphenyl)urea; and
1-[3-(morpholino-4-carbonyl)phenyl]-3-(4-trifluoromethylphenyl)urea.

### DESCRIPTION OF THE FIGURES

FIG. 1 shows a graph of body weight gain over time for mice on a high-fat, high-fructose diet administered with 20 mg/kg of compound 5, 60 mg/kg compound 5, or vehicle (control) by oral gavage twice daily. On Day 1, the animals were placed on high-fat, high-fructose diet. On week 5, the animals began being treated with vehicle or compound 5 at 20 and 60 mg/kg twice daily by oral gavage.

FIG. 2A shows a graphic intra-group comparison for the pre- and post-dose Glucose Tolerance Test (GTT) measurements for mice on a high-fat, high-fructose diet administered with 20 mg/kg of compound 5 by oral gavage twice daily following either 3 weeks or 5.5 weeks post initiation of dosing.

FIG. 2B shows a graphic intra-group comparison for the pre- and post-dose GTT measurements for mice on a high-fat, high-fructose diet administered with 60 mg/kg of compound 5 by oral gavage twice daily following either 3 weeks or 5.5 weeks post initiation of dosing.

FIG. 2C shows a graphic intra-group comparison for the pre- and post-dose GTT measurements for mice on a high-fat, high-fructose diet administered vehicle alone (control).

FIG. 2D shows a graphic comparison for the pre- and post-dose glucose area under the curve (AUC) measurements for mice on a high-fat, high-fructose diet administered with 20 mg/kg of compound, 60 mg/kg of compound, or vehicle by oral gavage twice daily.

FIG. 3A shows a graphic comparison for the GTT measurements for mice at 8 weeks on the high-fat, high-fructose diet and administered with 20 mg/kg of compound 5, 60 mg/kg of compound 5, or vehicle alone (control) by oral gavage twice daily for 3 weeks. The X-axis measures time in minutes after administration whereas the Y-axis measures the glucose serum level in mg/dL.

FIG. 3B shows a graphic comparison for the GTT measurements for mice at 10.5 weeks on a high-fat, high-fructose diet and administered with 20 mg/kg of compound 5, 60 mg/kg of compound 5, or vehicle alone (control) by oral gavage twice daily for 5.5 weeks.

FIGS. 4A, 4B, and 4C show bar graphs of systolic, diastolic, and mean blood pressure measurements, respectively, for mice after 8 weeks on a high-fat, high-fructose diet administered with 20 mg/kg of compound 5, 60 mg/kg compound 5, or vehicle (control) by oral gavage twice daily. FIG. 4D shows a bar graph of heart rate for mice after 8 weeks on a high-fat, high-fructose diet administered with 20 mg/kg of compound 5, 60 mg/kg compound 5, or vehicle (control) by oral gavage twice daily.

FIG. 5 shows a bar graph of serum cholesterol levels for mice after 5 weeks or 10 weeks (5 weeks of which are on the designated compound) on a high-fat, high-fructose diet administered with 20 mg/kg of compound 5, 60 mg/kg compound 5, or vehicle (control) by oral gavage twice daily.

FIG. 6 shows a graph of body weight change over time starting at week 8 for mice feed with either standard chow and water diet (NC) or high-fat, high-fructose diet (HF) following administration of vehicle (CMC-Tween), 10 mg/kg/day in drinking water of Losartan or with 60 mg/kg of compound 3, compound 4 or compound 5 twice daily by oral gavage.

FIG. 7 shows a graphic comparison of Glucose Tolerance Test (GTT) measurements for mice on either standard chow and water diet (NC) or high-fat, high-fructose diet (HF), following 4 weeks of administered with vehicle (CMC-Tween), 10 mg/kg/day in drinking water of Losartan or with 60 mg/kg of compound 3, compound 4 or compound 5 by oral gavage twice daily. The X-axis measures time in minutes after administration whereas the Y-axis measures the glucose serum level in mg/dL.

FIG. 8 shows a graphic comparison of serum cholesterol levels for mice on either standard chow and water diet (NC) or high-fat, high-fructose diet (HF) following 4 weeks administration with vehicle (CMC-Tween), 10 mg/kg/day in drinking water of Losartan or with 60 mg/kg of compound 3, compound 4 or compound 5 by oral gavage twice daily.

### DETAILED DESCRIPTION

Throughout this disclosure, various publications, patents and published patent specifications are referenced by an identifying citation. The disclosures of these publications, patents and published patent specifications are hereby incorporated by reference in their entirety into the present disclosure to more fully describe the state of the art to which this invention pertains.

As used herein, certain terms have the following defined meanings.

As used in the specification and claims, the singular form “a”, “an” and “the” include plural references unless the context clearly dictates otherwise.

“Cis-Epoxyeicosatrienoic acids” (“EETs”) are biomediators synthesized by cytochrome P450 epoxygenases.

“Epoxide hydrolases” (“EH;” EC 3.3.2.3) are enzymes in the alpha/beta hydrolase fold family that add water to 3 membered cyclic ethers termed epoxides.

“Soluble epoxide hydrolase” (“sEH”) is an enzyme which in endothelial, smooth muscle and other cell types converts EETs to dihydroxy derivatives called dihydroxyeicosatrienoic acids (“DHETs”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). The evolution and nomenclature of the gene is discussed in Beetham et al., DNA Cell Biol. 14(1):61-71 (1995). Soluble epoxide hydrolase represents a single highly conserved gene product with over 90% homology between rodent and human (Arand et al., FEBS Lett., 338:251-256 (1994)).

“sEH inhibitor” refers to an inhibitor that inhibits by 50% the activity of sEH in hydrolyzing epoxides at a concentration of less than about 500 μM, preferably, the inhibitor inhibits by 50% the activity of sEH in hydrolyzing epoxides at a concentration of less than about 100 μM, even more preferably, the inhibitor inhibits by 50% the activity of sEH in hydrolyzing epoxides at a concentration of less than about 100 nM, and most preferably, the inhibitor inhibits by 50% the activity of sEH in hydrolyzing epoxides at a concentration of less than about 50 nM.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkenyl” refers to straight or branched hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, preferably 1 to 3, or more preferably 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NR²⁰C(O)alkyl, —NR²⁰C(O)substituted alkyl, —NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR¹²R²² where R²¹ and R²² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cycloalkenyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²¹ and R²² are optionally joined, together with the nitrogen bound thereto to form a heterocyclic or substituted heterocyclic group, provided that R²¹ and R²² are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R²¹ is hydrogen and R²² is alkyl, the substituted amino group is sometimes referred to herein as alkylamino. When R²¹ and R²² are alkyl, the substituted amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Aminocarbonyl” refers to the group —C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²⁰C(O)NR¹⁰R¹¹ where R²⁰ is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR²⁰C(S)NR¹⁰R¹¹ where R²⁰ is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR²⁰—SO₂NR¹⁰R¹¹ where R²⁰ is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR¹²)NR¹⁰R¹¹ where R¹⁰, R¹¹, and R¹² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Carboxy” or “carboxyl” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR²⁰—C(O)O-alkyl, —NR²⁰—C(O)O-substituted alkyl, —NR²⁰—C(O)O-alkenyl, —NR²⁰—C(O)O-substituted alkenyl, —NR²⁰—C(O)O-alkynyl, —NR²⁰—C(O)O-substituted alkynyl, —NR²⁰—C(O)O-aryl, —NR²⁰—C(O)O-substituted aryl, —NR²⁰—C(O)O-cycloalkyl, —NR²⁰—C(O)O-substituted cycloalkyl, —NR²⁰—C(O)O-cycloalkenyl, —NR²⁰—C(O)O-substituted cycloalkenyl, —NR²⁰—C(O)O-heteroaryl, —NR²⁰—C(O)O-substituted heteroaryl, —NR²⁰—C(O)O-heterocyclic, and —NR—C(O)O-substituted heterocyclic wherein R²⁰ is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring. Examples of suitable cycloalkyl groups include, for instance, adamantyl, cyclopropyl, cyclobutyl, cyclopentyl, and cyclooctyl. Other examples of cycloalkyl groups include bicycle[2,2,2,]octanyl, norbornyl, and spiro groups such as spiro[4.5]dec-8-yl:

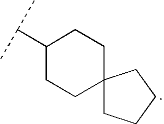

“Cycloalkenyl” refers to non-aromatic cyclic alkyl groups of from 3 to 10 carbon atoms having single or multiple cyclic rings and having at least one >C═C< ring unsaturation and preferably from 1 to 2 sites of >C═C< ring unsaturation.

“Substituted cycloalkyl” and “substituted cycloalkenyl” refers to a cycloalkyl or cycloalkenyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy” refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy” refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR¹³C(═NR¹³)N(R¹³)₂ where each R¹³ is independently selected from the group consisting of hydrogen, alkyl, substituted alkyl, aryl, substituted aryl, heteroaryl, substituted heteroaryl, heterocyclic, and substituted heterocyclic and two R¹³ groups attached to a common guanidino nitrogen atom are optionally joined together with the nitrogen bound thereto to form a heterocyclic or substituted heterocyclic group, provided that at least one R¹³ is not hydrogen, and wherein said substituents are as defined herein.

“Halo” or “halogen” refers to fluoro, chloro, bromo and iodo and preferably is fluoro or chloro.

“Haloalkyl” refers to alkyl groups substituted with 1 to 5, 1 to 3, or 1 to 2 halo groups, wherein alkyl and halo are as defined herein.

“Haloalkoxy” refers to alkoxy groups substituted with 1 to 5, 1 to 3, or 1 to 2 halo groups, wherein alkoxy and halo are as defined herein.

“Haloalkylthio” refers to alkylthio groups substituted with 1 to 5, 1 to 3, or 1 to 2 halo groups, wherein alkylthio and halo are as defined herein.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic ring. In one embodiment, the nitrogen and/or sulfur atom(s) of the heterocyclic group are optionally oxidized to provide for the N-oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Heterocyclyloxy” refers to the group —O-heterocyclyl.

“Substituted heterocyclyloxy” refers to the group —O-(substituted heterocyclyl).

“Heterocyclylthio” refers to the group —S-heterocyclyl.

“Substituted heterocyclylthio” refers to the group —S-(substituted heterocyclyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻).

“Spiro ring systems” refers to bicyclic ring systems that have a single ring carbon atom common to both rings.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—. The term “alkylsulfonyl” refers to —SO₂-alkyl. The term “(substituted sulfonyl)amino” refers to —NH(substituted sulfonyl) wherein substituted sulfonyl is as defined herein.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

“Stereoisomer” or “stereoisomers” refer to compounds that differ in the chirality at one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically-acceptable carrier” encompasses any of the standard pharmaceutical carriers, such as a phosphate-buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

An “excipient” refers to an inert substance added to a pharmaceutical composition to further facilitate administration of the active ingredient.

A “subject,” “individual” or “patient” is used interchangeably herein, and refers to a vertebrate, for example a mammal or preferably a human. Mammals include, but are not limited to, murines, rats, simians, humans, farm animals, sport animals and pets.

An “effective amount” is used synonymously with a “therapeutically effective amount” and intends an amount sufficient to effect beneficial or desired results. An effective amount can be administered in one or more administrations, applications, or dosages.

“Treating” or “treatment” of a disease or condition will depend on the disease or condition to be treated and the individual to be treated. In general, treatment intends one or more of (1) inhibiting the progression of the manifested disease or condition as measured by clinical or sub-clinical parameters (where the term “Inhibiting” or “Inhibition” is intended to be a subset of “Treating” or “treatment”), (2) arresting the development of the disease as measured by clinical or sub-clinical parameters, (3) ameliorating or causing regression of the disease or condition as measured by clinical or sub-clinical parameters, or (4) reducing pain or discomfort for the subject as measured by clinical parameters. “Treating” does not include preventing the onset of the disease or condition.

“Preventing” or “prevention” of a disease or condition means that the onset of the disease or condition in a subject predisposed thereto is prevented such that subject does not manifest the disease or condition.

### Therapeutic Methods

The present invention is directed to the use of sEH inhibitors to treat, prevent, or inhibit metabolic syndrome and conditions associated with metabolic syndrome. The present invention is further directed to the surprising and unexpected discovery that use of sEH inhibitors can beneficially reduce the risk in a subject of developing, or further developing, one or multiple conditions related to metabolic syndrome. Such conditions include, by way of example, glucose intolerance, elevated serum cholesterol or triglyceride levels, incipient diabetes, obesity, high blood pressure, and the like. Left untreated these conditions could lead to serious disorders such as diabetes, dyslipidemia, and cardiovascular disease. Early intervention with the methods described herein not only prevents or inhibits the onset of one or more of these conditions but, in many cases, actual reversal of the adverse condition or related disorder can be achieved.

It has previously been shown that sEH inhibitors can reduce hypertension. See e.g. U.S. Pat. No. 6,351,506. However, it was not known prior to the present invention that sEH inhibitors can be used to prevent or inhibit metabolic syndrome and to treat multiple conditions associated with the syndrome.

Metabolic syndrome is characterized by a group of metabolic risk factors present in one person. The metabolic risk factors include central obesity (excessive fat tissue in and around the abdomen), atherogenic dyslipidemia (blood fat disorders—mainly high triglycerides and low HDL cholesterol), insulin resistance or glucose intolerance, prothrombotic state (e.g., high fibrinogen or plasminogen activator inhibitor in the blood), and high blood pressure (130/85 mmHg or higher).

Metabolic syndrome, in general, can be diagnosed based on the presence of three or more of the following clinical manifestations in one subject:

a) Abdominal obesity characterized by a elevated waist circumference equal to or greater than 40 inches (102 cm) in men and equal to or greater than 35 inches (88 cm) in women or obesity characterized by a body mass index (BMI) equal to or greater than 25, or in another aspect a BMI equal to or greater than 30, or in another aspect a BMI equal to or greater than 35, or in yet another aspect a BMI equal to or greater than 40;

b) Elevated triglycerides equal to or greater than 150 mg/dL, or in one aspect equal to or greater than 200 mg/dL, or in another aspect less than 215 mg/dL, or in another aspect equal to or greater than 150 mg/dL but less than 200 mg/dL, or in yet another aspect equal to or greater than 150 mg/dL but less than 215 mg/dL;

c) Reduced levels of high-density lipoproteins (HDL) of less than 40 mg/dL in women and less than 50 mg/dL in men, or alternatively less than 35 mg/dL in women and less than 45 mg/dL in men, or alternatively less than 30 mg/dL in women and less than 40 mg/dL in men, or alternatively between 10 mg/dL to 40 mg/dL in women and between 10 mg/dL to 50 mg/dL in men, or alternatively between 15 mg/dL to 40 mg/dL in women and between 15 mg/dL to 50 mg/dL in men, or alternatively, between 20 mg/dL to 40 mg/dL in women and between 20 mg/dL to 50 mg/dL in men, or alternatively between 40 mg/dL to 50 mg/dL for both men and women;

d) High blood pressure equal to or greater than 130/85 mm Hg, or alternatively equal to or greater than 140/90, or alternatively equal to or greater than 150/90, or alternatively equal to or greater than 140/100, or alternatively equal to or greater than 150/100; and

e) Elevated fasting glucose equal to or greater than 100 mg/dL, or alternatively, equal to or greater than 110 mg/dL, or alternatively equal to or greater than 120, or alternatively equal to or greater than 100 mg/dL, but in all cases less than 125 mg/dL.

It is desirable to provide early intervention to prevent the onset of metabolic syndrome so as to avoid the medical complications brought on by this syndrome. Prevention or inhibition of metabolic syndrome refers to early intervention in subjects predisposed to, but not yet manifesting, metabolic syndrome. These subjects may have a genetic disposition associated with metabolic syndrome and/or they may have certain external acquired factors associated with metabolic syndrome, such as excess body fat, poor diet, and physical inactivity. Additionally, these subjects may exhibit one or more of the conditions associated with metabolic syndrome. These conditions can be in their incipient form.

Accordingly, one aspect, the invention provides a method for inhibiting the onset of metabolic syndrome by administering to the subject predisposed thereto an effective amount of a sEH inhibitor.

In another aspect, the invention provides a method for treating a mammalian subject suffering from metabolic syndrome by administration of an effective amount of one or more of the compounds described herein, wherein the metabolic syndrome is characterized by the presence of the clinical manifestations which are obesity, elevated triglycerides and high blood pressure as described above. Alternatively, the clinical manifestations are elevated triglycerides, reduced levels of high-density lipoproteins and high blood pressure as described above. In another aspect, the clinical manifestations are obesity, high blood pressure, and reduced high-density lipoproteins as described above. In yet another aspect, the clinical manifestations are elevated triglycerides, obesity, and reduced high-density lipoproteins as described above. In yet another aspect, the clinical manifestations are reduced levels of high-density lipoproteins, high blood pressure, and elevated fasting glucose as described above.

In another aspect, the invention provides a method for treating a mammalian subject suffering from metabolic syndrome by administration of an effective amount of one or more of the compounds described herein, wherein the metabolic syndrome is characterized by the presence of any of the combinations described in Table 1 selected from:

a) Abdominal obesity;

b) Elevated triglycerides;

c) Reduced levels of high-density lipoproteins (HDL);

d) High blood pressure; and

e) Elevated fasting,

as described above.

TABLE 1
Combinations of clinical manifestations for diagnosing Metabolic
syndrome

| Combination No. | Clinical Manifestations |
|---|---|
| 1. | a, b, and c |
| 2. | a, b, and d |
| 3. | a, b, and e |
| 4. | a, c, and d |
| 5. | a, c, and e |
| 6. | a, d, and e |
| 7. | b, c, and d |
| 8. | b, c, and e |
| 9. | b, d, and e |
| 10. | c, d, and e |
| 11. | a, b, c, and d |
| 12. | a, b, c, and e |
| 13. | a, c, d, and e |
| 14. | b, c, d, and e |
| 15. | a, b, d, and e |
| 16. | a, b, c, d, and e |

Another aspect provides a method for treating one or more conditions associated with metabolic syndrome in a subject where the conditions are selected from incipient diabetes, obesity, glucose intolerance, high blood pressure, elevated serum cholesterol, reduced high-density lipoproteins, and elevated triglycerides. This method comprises administering to the subject an amount of a sEH inhibitor effective to treat the condition or conditions manifested in the subject. In one embodiment of this aspect, two or more of the noted conditions are treated by administering to the subject an effective amount of a sEH inhibitor. In this aspect, the conditions to be treated include treatment of hypertension. In another aspect, the methods of the invention are useful for improving serum levels of low-density lipoproteins (LDL) and/or high-density lipoproteins (HDL). In further aspect, the methods of the invention are useful for decreasing serum LDL. In yet a further aspect, the methods of the invention are useful for increasing serum HDL.

sEH inhibitors are also useful in treating metabolic conditions comprising obesity, glucose intolerance, reduced high-density lipoproteins, hypertension, high blood pressure, elevated levels of serum cholesterol, and elevated levels of triglycerides, or combinations thereof, regardless if the subject is manifesting, or is predisposed to, metabolic syndrome.

Accordingly, another aspect of the invention provides for methods for treating a metabolic condition in a subject, comprising administering to the subject an effective amount of a sEH inhibitor, wherein the metabolic condition is selected from the group consisting of conditions comprising obesity, glucose intolerance, high blood pressure, elevated serum cholesterol, reduced high-density lipoproteins, and elevated triglycerides, and combinations thereof.

In a further aspect of the above embodiments, a mammalian subject suffering from metabolic syndrome or metabolic conditions is not suffering from nephropathy. In a further aspect, the mammalian subject of the above embodiments does not have nephropathy associated with metabolic syndrome or diabetes mellitus. In yet a further aspect, the compounds of the invention are not for inhibiting development or progression of nephropathy.

In general, levels of glucose, serum cholesterol, triglycerides, obesity, and blood pressure are well known parameters and are readily determined using methods known in the art.

Several distinct categories of glucose intolerance exist, including for example, type 1 diabetes mellitus, type 2 diabetes mellitus, gestational diabetes mellitus (GDM), impaired glucose tolerance (IGT), and impaired fasting glucose (IFG). IGT and IFG are transitional states from a state of normal glycemia to diabetes. IGT is defined as two-hour glucose levels of 140 to 199 mg per dL (7.8 to 11.0 mmol) on the 75-g oral glucose tolerance test (OGTT), and IFG is defined as fasting plasma glucose (FG) values of 100 to 125 mg per dL (5.6 to 6.9 mmol per L) in fasting patients. These glucose levels are above normal but below the level that is diagnostic for diabetes. Rao, et al., Amer. Fam. Phys. 69:1961-1968 (2004).

Current knowledge suggests that development of glucose intolerance or diabetes is initiated by insulin resistance and is worsened by the compensatory hyperinsulinemia. The progression to type 2 diabetes is influenced by genetics and environmental or acquired factors including, for example, a sedentary lifestyle and poor dietary habits that promote obesity. Patients with type 2 diabetes are usually obese, and obesity is also associated with insulin resistance.

“Incipient diabetes” refers to a state where a subject has elevated levels of glucose or, alternatively, elevated levels of glycosylated hemoglobin, but has not developed diabetes. A standard measure of the long term severity and progression of diabetes in a patient is the concentration of glycosylated proteins, typically glycosylated hemoglobin. Glycosylated proteins are formed by the spontaneous reaction of glucose with a free amino group, typically the N-terminal amino group, of a protein. HbA1c is one specific type of glycosylated hemoglobin (Hb), constituting approximately 80% of all glycosylated hemoglobin, in which the N-terminal amino group of the Hb A beta chain is glycosylated.

Formation of HbA1c irreversible and the blood level depends on both the life span of the red blood cells (average 120 days) and the blood glucose concentration. A buildup of glycosylated hemoglobin within the red cell reflects the average level of glucose to which the cell has been exposed during its life cycle. Thus the amount of glycosylated hemoglobin can be indicative of the effectiveness of therapy by monitoring long-term serum glucose regulation. The HbA1c level is proportional to average blood glucose concentration over the previous four weeks to three months. Therefore HbA1c represents the time-averaged blood glucose values, and is not subject to the wide fluctuations observed in blood glucose values, a measurement most typically taken in conjunction with clinical trials of candidate drugs for controlling diabetes. In one embodiment, HbA1c levels of greater than 6 and less than 7 are typically associated with incipient diabetes.

Obesity can be monitored by measuring the weight of a subject or by measuring the Body Mass Index (BMI) of a subject as described in “Clinical Guidelines on the Identification Evaluation and Treatment of overweight and obesity in Adults” The Evidence Report, NIH Publication No. 98-4083, September 1998. BMI is determined by dividing the subject's weight in kilograms by the square of his/her height in meters (BMI=kg/m2). Alternatively, obesity can be monitored by measuring percent body fat. Percent body fat can be measured by methods known in the art including by weighing a subject underwater, by a skin fold test, in which a pinch of skin is precisely measured to determine the thickness of the subcutaneous fat layer, or by bioelectrical impedance analysis. In one aspect of the invention, obesity is characterized by a BMI equal to or greater than 25, or in another aspect a BMI equal to or greater than 30, or in another aspect a BMI equal to or greater than 35, or in yet another aspect a BMI equal to or greater than 40.

### sEH Inhibitors

In each of the above embodiments, an effective amount of a sEH inhibitor, or composition comprising a sEH inhibitor, is administered to a subject in need thereof. Preferably, the sEH inhibitors are described by at least one of the following general or specific formulas shown in Formula (I), Formula (II), or Formula (IIa).

In one aspect, the compound is a member of the group of Formula (I):

R¹NHC(═O)NHR² (I)

wherein:
Q is selected from the group consisting of O and S; and
R¹ and R² are independently selected from the group consisting of substituted alkyl, aryl, substituted aryl, heteroaryl, substituted heteroraryl, cycloalkyl, substituted cycloalkyl, heterocycloalkyl, and substituted heterocycloalkyl.

In one aspect, the compound is a member of the group of Formula (II):

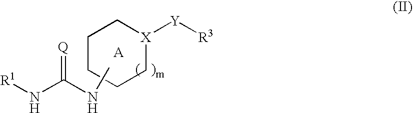

wherein:
Q is selected from the group consisting of O and S;
R¹ is selected from the group consisting of aryl, substituted aryl, heteroaryl, substituted heteroraryl, cycloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂;
R³ is selected from the group consisting of alkyl, substituted alkyl, or heterocycloalkyl; and
m is selected from the group consisting of zero, 1, and 2.

In one aspect, the compound is a member of the group of Formula (IIa):

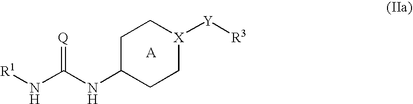

wherein:
Q is selected from the group consisting of O and S;
R¹ is selected from the group consisting of substituted aryl, substituted aryl, heteroaryl, substituted heteroraryl, cycloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂; and
R³ is selected from the group consisting of alkyl, substituted alkyl, or heterocycloalkyl.

In some aspects, Q is O.

In some aspects, R¹ is phenyl optionally substituted with one to three groups independently selected from halo, alkyl, acyl, acyloxy, carboxyl ester, acylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, and alkylsulfonyl.

In some aspects, R¹ is selected from the group consisting of 4-trifluoromethylphenyl or 4-trifluoromethoxyphenyl.

In some aspects, R¹ is cycloalkyl. In some such aspects, R¹ is adamantyl.

In other aspects, R³ is alkyl. In some such aspects, R³ is methyl.

In other aspects, R³ is heterocycloalkyl. In some such aspects, R³ is morpholino.

In another embodiment, the compound to be administered is a compound, stereoisomer, or a pharmaceutically acceptable salt thereof a compound selected from Table 2.

TABLE 2

| Compound | |
|---|---|
| No. | Name |
| 1 | 1-[3-(morpholino-4-carbonyl)phenyl]-3-(4- |
| | trifluoromethylphenyl) urea |
| 2 | 1-[1-(acetyl)piperidin-4-yl]-3-(adamant-1-yl) urea |
| 3 | 1-[1-(acetyl)piperidin-4-yl]-3-(4-trifluoromethylphenyl) |
| | urea |
| 4 | 1-[1-(methylsulfonyl)piperidin-4-yl]-3-(4- |
| | trifluoromethylphenyl) urea |
| 5 | 1-[1-(methylsulfonyl)piperidin-4-yl]-3-(adamant-1-yl) urea |

In another aspect of the invention, one or more of the compounds of Formula (I), (II), or (IIa) or pharmaceutically acceptable salts thereof, may be used in the preparation of a medicament for the treatment of metabolic syndrome or metabolic conditions selected from one or more of the following: incipient diabetes, obesity, glucose intolerance, high blood pressure, elevated serum cholesterol, reduced high-density lipoproteins, or elevated triglycerides.

### Compositions and Formulations

The compositions are comprised of, in general, a sEH inhibitor in combination with at least one pharmaceutically acceptable carrier or excipient. Acceptable carriers are known in the art and described supra. Acceptable excipients are non-toxic, aid administration, and do not adversely affect the therapeutic benefit of the compound. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols.

The sEH inhibitors can be administered in any suitable formulation such as a tablet, pill, capsule, semisolid, gel, transdermal patch or solution, powders, sustained release formulation, solution, suspension, elixir or aerosol. The most suitable formulation will be determined by the disease or disorder to be treated and the individual to be treated.

Compressed gases may be used to disperse a sEH inhibitor of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The following are representative pharmaceutical formulations containing a sEH inhibitor of the present invention.

### Tablet Formulation

The following ingredients are mixed intimately and pressed into single scored tablets.

| Ingredient | Quantity per tablet, mg |
|---|---|
| sEH inhibitor | 400 |
| Cornstarch | 50 |
| Croscarmellose sodium | 25 |
| Lactose | 120 |
| Magnesium stearate | 5 |

### Capsule Formulation

The following ingredients are mixed intimately and loaded into a hard-shell gelatin capsule.

| Ingredient | Quantity per capsule, mg |
|---|---|
| sEH inhibitor | 200 |
| Lactose, spray-dried | 148 |
| Magnesium stearate | 2 |

### Suspension Formulation

The following ingredients are mixed to form a suspension for oral administration (q.s.=sufficient amount).

| Ingredient | Amount | |
|---|---|---|
| sEH inhibitor | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.0 | g |
| Veegum K (Vanderbilt Co) | 1.0 | g |
| Flavoring | 0.035 | mL |
| colorings | 0.5 | mg |
| distilled water | q.s. to 100 | mL |

### Injectable Formulation

The following ingredients are mixed to form an injectable formulation.

| | Quantity per injection, |
|---|---|
| Ingredient | mg |
| sEH inhibitor | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| | Quantity per |
|---|---|
| Ingredient | suppository, mg |
| sEH inhibitor | 500 mg |
| Witepsol ® H-15 | balance |

Also provided is a medicament comprising a compound or composition as described herein for use in treating a disease or disorder as described above, which can be identified by noting any one or more clinical or sub-clinical parameters.

### Combination Therapy

Because of the very nature of metabolic syndrome, it is often treated with combinations of agents where each is intended to impact one of the aspects of the disease. For more generalized therapeutic purposes, combination therapy is often desirable. Combination therapy includes administration of a single pharmaceutical dosage formulation which contains a sEH inhibitor and one or more additional active agents, or therapies such as heat, light and such, as well as administration of the sEH inhibitor and each active agent in its own separate pharmaceutical dosage formulation. For example, a compound of this invention and one or more of other agents such as angiotensin converting enzyme (ACE) inhibitors such as captopril or enalapril which are known to lower blood pressure and the like, and a HMG-CoA reductase inhibitor or statin such as atorvastatin or fluvastatin which lowers plasma cholesterol could be administered to the human subject together in a single oral dosage composition such as a tablet or capsule or each agent can be administered in separate oral dosage formulations. Other useful agents in the treatment of the individual components of metabolic syndrome include insulin sensitizers such as thiazolidinones also known a glitazones (examples: rosiglitazone, pioglitazone) and metformin. Agents that lower blood pressure include ACE inhibitors (examples: captipril, quinapril), angiotensinII receptor antagonists (examples: losartan, candesartan, olmesartan), beta blockers (examples: propranolol, metaprolol, atenolol), diuretics (examples: furosamide, hydrochlorothiazide), and calcium channel blockers (examples: nitrendipine, nicardapine, felodipine, verapamil, diltiazem). Agents known to impact the dislipidimia include fibrates (examples: chlofibrate, gemfibrate). Agents known to decrease plasma cholesterol include statins (examples: atorvastatin, fluvastatin, lovastatin, simvastatin) and niacin. Combination therapy is understood to include all these regimens.

### Dosing and Administration

The present invention provides therapeutic methods generally involving administering to a subject in need thereof an effective amount of sEH inhibitors described herein. The dose, frequency, and timing of such administering will depend in large part on the selected therapeutic agent, the nature of the condition to be treated, the condition of the subject, including age, weight and presence of other conditions or disorders, the formulation of the therapeutic agent and the discretion of the attending physician. The sEH inhibitors and compositions described herein and the pharmaceutically acceptable salts thereof are administered via oral, parenteral, subcutaneous, intramuscular, intravenous or topical routes. Generally, it is contemplated that the sEH inhibitors are to be administered in dosages ranging from about 0.10 milligrams (mg) up to about 1000 mg per day, although variations will necessarily occur, depending, as noted above, on the target tissue, the subject, and the route of administration. In preferred embodiments, the sEH inhibitors are administered orally once or twice a day.

The sEH inhibitors are preferably administered in a range between about 0.10 mg and 1000 mg per day, more preferably the compounds are administered in a range between about 1 mg and 800 mg per day; more preferably, the compounds are administered in a range between about 2 mg and 600 mg per day; more preferably, the compounds are administered in a range between about 5 mg and 500 mg per day; yet more preferably, the compounds are administered in a range between about 10 mg and 200 mg per day; yet even more preferably, the compounds are administered in a range between about 50 mg and 100 mg per day.

The following examples are provided to illustrate certain aspects of the present invention and to aid those of skill in the art in practicing the invention. These examples are in no way to be considered to limit the scope of the invention.

### Synthetic Chemistry

The sEH inhibitors of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the sEH inhibitors of this invention may contain one or more chiral centers. Accordingly, if desired, such inhibitors can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Preferably, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds of the invention may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

Scheme 1 below illustrates a general synthetic method for the preparation of the compounds of formula I.

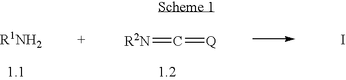

A synthesis of the compounds of the invention is shown in Scheme 1, where Q, R¹, and R² are as previously defined. Specifically, amine 1.1 reacts with the appropriate isocyanate 1.2 to form the corresponding urea or thiourea of formula I. Typically, the formation of the urea is conducted using a polar solvent such as DMF (dimethylformamide) at 0 to 10° C. Isocyanate or thioisocyanate 1.2 can be either known compounds or can be prepared from known compounds by conventional synthetic procedures. Suitable isocyanates include by way of example only, adamantyl isocyanate, cyclohexyl isocyanate, phenyl isocyanate, trifluoromethylphenyl isocyanate, chlorophenyl isocyanate, fluorophenyl isocyanate, trifluoromethoxyphenyl isocyanate and the like.

Scheme 2 illustrates the methods of Scheme 1 as they relate to the preparation of piperidinyl urea compounds of Formula (I).

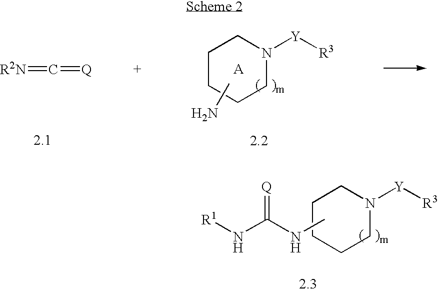

Scheme 2 can also be employed for the synthesis of compounds of formula (II) where, for illustrative purposes, ring A is a piperidinyl ring and Q, Y, R¹, R³, and m are previously defined. Reaction of isocyanate 2.1 with amine 2.2 forms the corresponding urea or thiourea of 2.3.

In Scheme 2, the N—(YR³) substituted piperidinyl amine can be prepared as shown in Scheme 3 below:

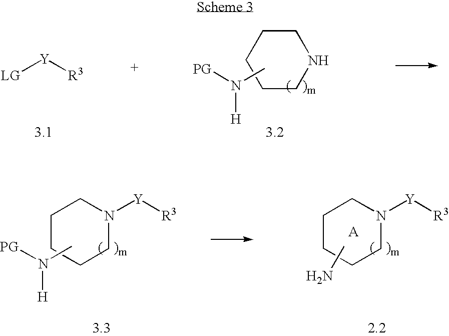

Where Y and R³ are as defined above and LG is a leaving group such as a halo group, a tosyl group, a mesyl group, and the like and PG is a conventional amino protecting group such as a tert-butoxycarbonyl (Boc) group. Reaction of 3.1 with protected aminopiperidine 3.2 forms the functionalized amine 3.3. Removal of the protecting group gives 2.2. Both of these reactions are conventional and well within the skill of the art.

The following schemes illustrate preferred methods of preparing compounds of Formula I and/or II. Specifically, in Scheme 4, a 4-amidopiperidine group is employed for illustrative purposes only and this scheme illustrates the synthesis of N-(1-acylpiperidin-4-yl)-N′-(adamant-1-yl)urea compounds:

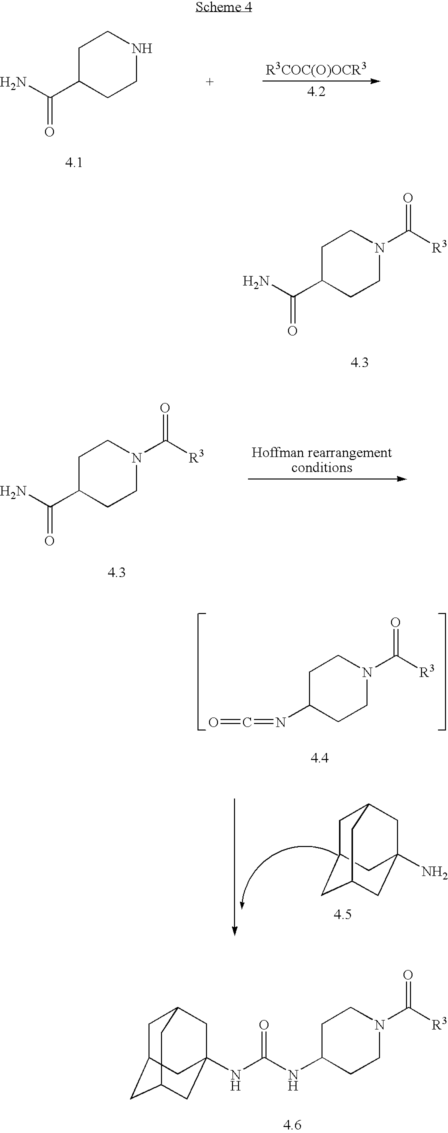

where R³ is defined herein.

In Scheme 4, the amino group of compound 4.1 is acylated using conventional conditions. Specifically, a stoichiometric equivalent or slight excess of a carboxylic acid anhydride 4.2 (which is used only for illustrative purposes) is reacted with compound 4.1 in the presence of a suitable inert diluent such as tetrahydrofuran, chloroform, methylene chloride and the like. When an acid chloride is employed in place of the acid anhydride, the reaction is typically conducted in the presence of an excess of a suitable base to scavenge the acid generated during the reaction. Suitable bases are well known in the art and include, by way of example only, triethylamine, diisopropylethylamine, pyridine, and the like.

The reaction is typically conducted at a temperature of from about 0 to about 40° C. for a period of time sufficient to effect substantial completion of the reaction which typically occurs within about 1 to about 24 hours. Upon reaction completion, the acylpiperidylamide, compound 4.3, can be isolated by conventional conditions such as precipitation, evaporation, chromatography, crystallization, and the like or, alternatively, used in the next step without isolation and/or purification. In certain cases, compound 4.3 precipitates from the reaction.

Compound 4.3 is then subjected to Hoffman rearrangement conditions to form isocyanate compound 4.4 under conventional conditions. In certain cases, Hoffman rearrangement conditions comprise reacting with an oxidative agent preferably selected from (diacetoxyiodo)benzene, base/bromine, base/chlorine, base/hypobromide, or base/hypochloride. Specifically, approximately stoichiometric equivalents of the N-acyl-4-amidopiperidine, compound 4.4, and, e.g., (diacetoxyiodo)benzene are combined in the presence of a suitable inert diluent such as acetonitrile, chloroform, and the like. The reaction is typically conducted at a temperature of from about 40° C., to about 100° C., and preferably at a temperature of from about 70° C., to about 85° C., for a period of time sufficient to effect substantial completion of the reaction which typically occurs within about 0.1 to about 12 hours. Upon reaction completion, the intermediate isocyanate, compound 4.4, can be isolated by conventional conditions such as precipitation, evaporation, chromatography, crystallization, and the like.

Alternatively and preferably, this reaction is conducted in the presence of adamantyl amine, compound 4.5, such that upon formation of the isocyanate, compound 4.4, the isocyanate functionality of this compound can react in situ with the amino functionality of compound 4.5 to provide for compound 4.6. In this embodiment, the calculated amount of the intermediate isocyanate is preferably employed in excess relative to the adamantyl amine and typically in an amount of from about 1.1 to about 1.2 equivalents based on the number of equivalents of adamantyl amine employed. The reaction conditions are the same as set forth above and the resulting product can be isolated by conventional conditions such as precipitation, evaporation, chromatography, crystallization, and the like.

Compound 4.4 is a stable intermediate. In certain cases, compound 4.3 is formed substantially free from impurities. Hence, Scheme 4 can be run as telescoping reaction processes.

Scheme 5 below illustrates an alternative synthesis of a urea compound where again a 4-amidopiperidine is employed for illustrative purposes:

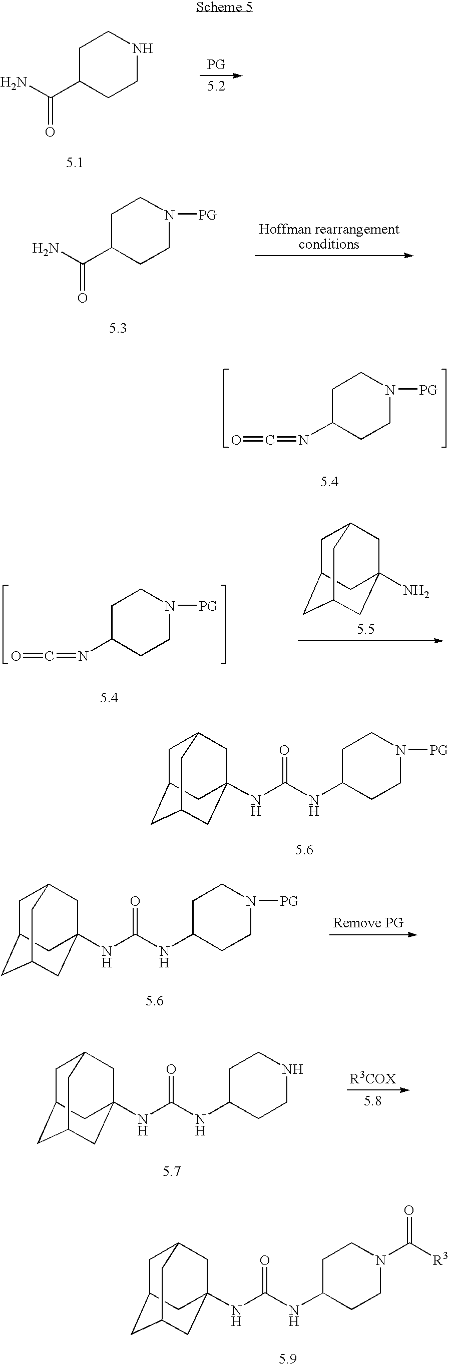

where R³ and PG are as defined herein and X is selected from the group consisting of OH, halo and —OC(O)R³.

Specifically, in Scheme 5, coupling of the adamantyl urea to the piperidinyl ring occurs prior to acylation of the piperidinyl nitrogen atom. In Scheme 5, the amine functionality of compound 5.1 is protected using a conventional amino protecting group (PG) which is well known in the art. In certain cases, the amino protecting group is a benzyl protecting group which can be derived from benzyl chloride and benzyl bromide. Compound 5.3 is subjected to Hoffman rearrangement conditions to form isocyanate compound 5.4 in the manner described in detail above. Compound 5.4 is a stable intermediate. The reaction of compound 5.4 with adamantyl amine is conducted as per Scheme 4 and is preferably conducted in a single reaction step wherein intermediate compound 5.4 is reacted in situ with adamantyl amine, compound 5.5, to form compound 5.6. Compound 5.6 is subjected to conditions to remove the protecting group to yield compound 5.7. In certain cases, the protecting group is benzyl and the removal conditions are palladium-carbon with methanol and formic acid. Compound 5.7 is acylated with compound 5.8 to form compound 5.9 as per Scheme 4 above.

Scheme 6 below illustrates the synthesis of N-(1-alkylsulfonylpiperidin-4-yl)-N′-(adamant-1-yl)ureas:

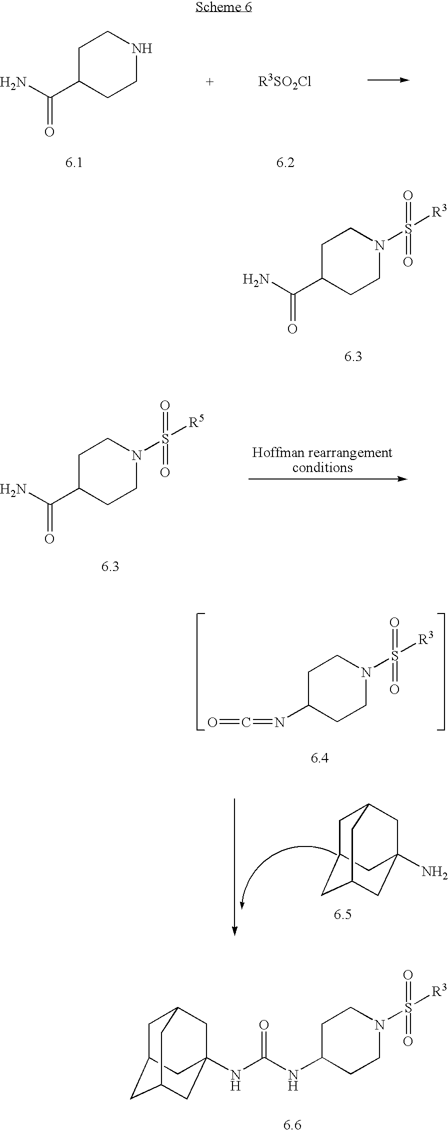

wherein R³ is defined herein.

Specifically, in Scheme 6, amino compound 6.1 is reacted with a sulfonyl halide, compound 6.2 (used for illustrative purposes only), to provide for sulfonamide compound 6.3. This reaction is typically conducted by reacting the compound 6.1 with at least one equivalent, preferably about 1.1 to about 2 equivalents, of the sulfonyl halide (for illustrative purposes depicted as the sulfonyl chloride) in an inert diluent such as dichloromethane, chloroform and the like. Generally, the reaction is preferably conducted at a temperature ranging from about −10° C. to about 20° C. for about 1 to about 24 hours. Preferably, this reaction is conducted in the presence of a suitable base to scavenge the acid generated during the reaction. Suitable bases include, by way of example, tertiary amines, such as triethylamine, diisopropylethylamine, N-methylmorpholine and the like. Alternatively, the reaction can be conducted under Schotten-Baumann-type conditions using aqueous alkali, such as sodium hydroxide and the like, as the base. Upon completion of the reaction, the resulting sulfonamide, compound 6.3, is recovered by conventional methods including neutralization, extraction, precipitation, chromatography, filtration, and the like or, alternatively, used in the next step without purification and/or isolation.

Compound 6.3 is subjected to Hoffman rearrangement conditions as described above to form isocyanate compound 6.4. The reaction of compound 6.4 with adamantyl amine, compound 6.5, is conducted as per Scheme 4 and is preferably conducted in a single reaction step wherein the isocyanate, compound 6.4, is reacted in situ with adamantyl amine, compound 6.5, to form compound 6.6.

The sulfonyl chlorides employed in the above reaction are also either known compounds or compounds that can be prepared from known compounds by conventional synthetic procedures. Such compounds are typically prepared from the corresponding sulfonic acid, using phosphorous trichloride and phosphorous pentachloride. This reaction is generally conducted by contacting the sulfonic acid with about 2 to 5 molar equivalents of phosphorous trichloride and phosphorous pentachloride, either neat or in an inert solvent, such as dichloromethane, at temperature in the range of about 0° C. to about 80° C. for about 1 to about 48 hours to afford the sulfonyl chloride. Alternatively, the sulfonyl chloride can be prepared from the corresponding thiol compound, i.e., from compounds of the formula R³—SH where R³ is as defined herein, by treating the thiol with chlorine (Cl₂) and water under conventional reaction conditions.

Compound 6.4 is a stable intermediate. In certain cases, compound 6.3 is formed substantially free from impurities. Hence Scheme 6 can be run as a telescoping reaction processes.

Scheme 7 below illustrates an alternative synthesis of a urea compound.

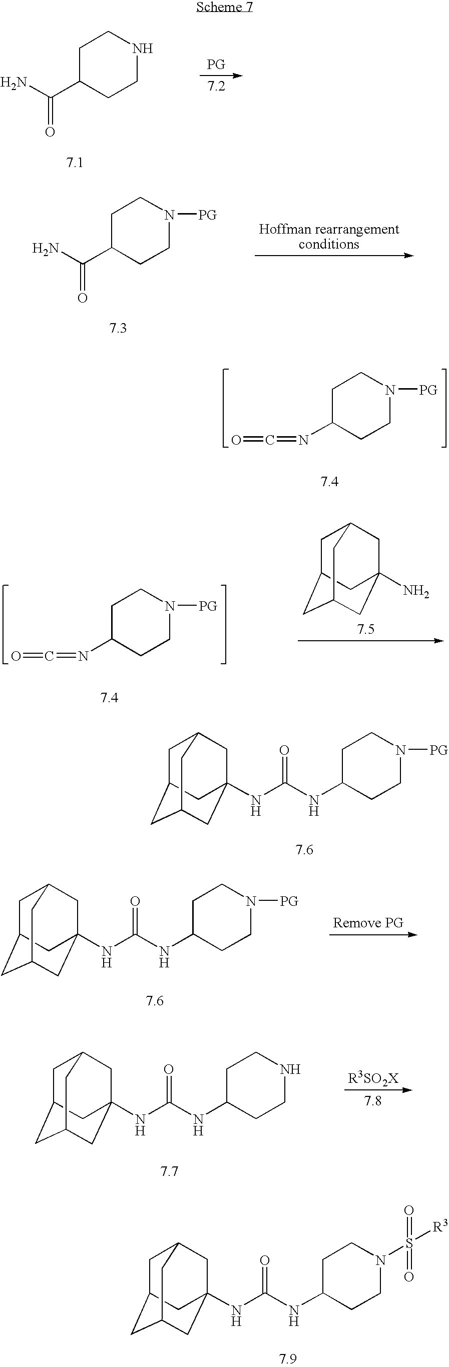

wherein R³, X and PG are defined herein.

Specifically, in Scheme 7, coupling of the adamantyl urea, compound 7.5, to the piperidinyl ring occurs prior to sulfonylation of the piperidinyl nitrogen atom. In Scheme 7, the amine functionality of compound 7.1 is protected using a conventional amino protecting group (PG) which are well known in the art. In certain cases, the amino protecting group is a benzyl protecting group which can be derived from benzyl chloride or benzyl bromide. Compound 7.3 is subjected to Hoffman rearrangement conditions to form isocyanate compound 7.4 in the manner described in detail above. Compound 7.4 is a stable intermediate. The reaction of compound 7.4 with adamantyl amine, compound 7.5, is conducted as per Scheme 4 and is preferably conducted in a single reaction step wherein intermediate compound 7.4 is reacted in situ with adamantyl amine, compound 7.5, to form compound 7.6. Compound 7.6 is subjected to conditions to remove the protecting group to yield compound 7.7. In certain cases, the protecting group is benzyl and the removal conditions are palladium-carbon with methanol and formic acid. Compound 7.7 is then sulfonylated with compound 7.8 to form compound 7.9 as per Scheme 6 above.

A further elaboration of processes suitable for preparing compounds of Formula (I), Formula (II) and Formula (IIa) are disclosed in Gless, U.S. patent application Ser. No. 12/021,090, filed on Jan. 28, 2008, which claims the benefit under 35 U.S.C. § 119(e) of U.S. Provisional Patent Application Nos. 60/887,114 filed on Jan. 29, 2007 and 60/972,177 filed on Sep. 13, 2007, all of which applications are incorporated herein in their entirety.

The following examples are provided to illustrate certain aspects of the present invention and to aid those of skill in the art in practicing the invention. These examples are in no way to be considered to limit the scope of the invention.

### EXAMPLES

The examples below as well as throughout the application, the following abbreviations have the following meanings. If not defined, the terms have their generally accepted meanings.
aq.=aqueous
bd=broad doublet
bm=broad multiplet
brs=broad singlet
bt=broad triplet
Boc=tert-Butoxycarbonyl
d=doublet
DCM=dichloromethane
DMAP=dimethylaminopyridine
DMF=dimethylformamide
DMSO=dimethylsulfoxide
eq.=equivalents
EtOAc=ethyl acetate
g=gram
HPLC=high performance liquid chromatography
LCMS=liquid chromatography mass spectroscopy
m=multiplet
M=molar
mg=milligram
MHz=megahertz
mL=milliliter
dL=deciliter
mM=millimolar
mmol=millimole
m.p.=melting point
MS=mass spectroscopy
N=normal
NMR=nuclear magnetic resonance
s=Singlet
t=Triplet
TLC=thin layer chromatography
μL=microliters

In all cases in the following examples the designation “Compound X”, where X is a number from 1 to 5, refers to the compounds as identified in Table 2 above.

### Example 1

### Synthesis of 1-[1-(acetyl)piperidin-4-yl]-3-(4-trifluoromethylphenyl)urea (Compound 3)

### Preparation of tert-butyl 4-aminopiperidine-1-carboxylate

4-Aminopiperidine (5.0 g, 50 mmol, 1 eq.) was added to a solution of benzaldehyde (5.1 mL, 50 mmol, 1 eq.) in toluene (130 mL) in a 250 mL 3-necked flask fitted with a Dean-Stark trap and a condenser. A nitrogen line was connected to the top of the condenser, and the reaction was refluxed for 3 hours, during which time, water was seen to condense in the Dean-Stark trap. The reaction was cooled to room temperature and Boc anhydride (5.8 mL, 50 mmol, 1 eq.) was added over 5 minutes. The reaction was stirred over night under a blanket of N₂. The solvent was then removed under vacuum and NaHSO₄ (1M in water, 50 mL) was added to the residue. The resulting mixture was stirred vigorously for 2 hours before partitioned between diethyl ether (250 mL) and water (250 mL). The aqueous layer was separated, washed with diethyl ether (3×150 mL) and basified with NaOH solution until the pH was approximately 11. The resulting solution was extracted with DCM (4×200 mL). The combined organic layer was dried over Na₂SO₄, filtered, and evaporated to give 8.0 g of tert-butyl 4-aminopiperidine-1-carboxylate as a yellow oil.

### Preparation of tert-butyl 4-(3-(4-(trifluoromethyl)phenyl)ureido)piperidine-1-carboxylate

4-trifluoromethylphenyl isocyanate (1.0 eq.) was added to a solution of tert-butyl 4-aminopiperidine-1-carboxylate (1 eq.) in ethanol (10 volumes). The reaction mixture was stirred overnight at 50° C. The solvent was removed under vacuum and the crude product was crystallized in diethyl ether to give tert-butyl 4-(3-(4-(trifluoromethyl)phenyl)ureido)piperidine-1-carboxylate as a white solid.

### Preparation of 1-(piperidin-4-yl)-3-(4-trifluoromethylphenyl)urea

tert-Butyl 4-(3-(4-(trifluoromethyl)phenyl)ureido)piperidine-1-carboxylate was stirred in MeOH/HCl overnight. The solvent was removed and the residue was stirred in diethyl ether until a white solid precipitate was seen. The precipitate was collected by filtration to give 1-(piperidin-4-yl)-3-(4-(trifluoromethyl)phenyl)urea as the hydrochloride salt.

### Preparation of 1-(1-acetyl-piperidin-4-yl)-3-(4-trifluoromethyl-phenyl)-urea (Compound 3)

To a solution of 1-(piperidin-4-yl)-3-(4-(trifluoromethyl)phenyl)urea (10.3 g, 35.8 mmol) in DCM (150 mL) cooled with an ice water bath was added sequentially Et₃N (14.9 mL, 107 mmol) and acetic anhydride (5.0 mL, 53.8 mmol). After stirring at room temperature for 18 hours, the resulting precipitate was filtered, washed with DCM (2×50 mL), dried under a high vacuum for 4 hours to give 1-(1-acetyl-piperidin-4-yl)-3-(4-trifluoromethyl-phenyl)-urea as a white solid (8.4 g, 71%). HPLC purity 99.0%; m.p.: 240-248° C.; MS: 330 [M+H]⁺;

¹H NMR (300 MHz, DMSO-d₆) δ: 8.79 (s, 1H, NH), 7.62-7.48 (m, 4H), 6.18 (d, 1H, J=7.5 Hz, NH), 4.11 (d, J=15 Hz, 1H), 3.89-3.72 (m, 2H), 3.08 (t, 1H), 2.91 (m, 1H), 1.99 (s, 3H), 1.85-1.77 (m, 2H), 1.45-1.07 (m, 2H).

### Example 2

### Preparation of 1-[1-(methylsulfonyl)piperidin-4-yl]-3-(4-trifluoromethylphenyl)urea (Compound 4)

To a solution of 1-(piperidin-4-yl)-3-(4-(trifluoromethyl)phenyl)urea (10.8 g, 37.6 mmol) (prepared as above) in DCM (150 mL) cooled with an ice water bath was added sequentially Et₃N (15.7 mL, 113 mmol) and methanesulfonyl chloride (4.37 mL, 56.4 mmol). The reaction was stirred at room temperature for 18 hours. Water (200 mL) was added and the mixture was stirred for another 18 hours. The resulting precipitate was collected by filtration, washed with water (2×50 mL), and dried for 18 hours to give the titled product (3.6 g). The supernatant from the filtration was phase separated. The organic layer was dried over Na₂SO₄, filtered, and concentrated to give an additional 4.0 g of product. The combined crude product (7.6 g) was recrystallized from EtOAc to give the pure product as a white solid (3.15 g, 23%). HPLC purity 93.8%; MS: 366 [M+H]⁺;

¹H NMR (300 MHz, CDCl₃+DMSO-d₆): δ 8.03 (s, 1H, NH), 7.12-7.00 (m, 4H), 5.86 (s, 1H), 3.37-3.20 (m, 3H), 2.95-2.82 (m, 1H), 2.58-2.41 (m, 4H), 1.72-1.58 (m, 2H), 1.24-1.08 (m, 2H).

### Example 3

### Preparation of 1-[3-(morpholino-4-carbonyl)phenyl]-3-(4-trifluoromethylphenyl)urea (Compound 1)

A solution of 4-trifluoromethylphenyl isocyanate (350 mg, 1.87 mmol) and 3-amino-benzoic acid (450 mg, 3.28 mmol) in DMF (10 mL) was warmed at 70° C. overnight. The reaction was monitored by TLC. The reaction mixture was cooled to room temperature and water (5 mL) and 1 N aq. HCl (5 mL) was added with ice bath cooling and stirred for 1 hour. The resulting solid was filtered, washed with water, hexane and dried in a vacuum oven. The crude product was recrystallized from acetone/hexane to afford 310 mg (51%) of product as a white solid. m.p. 271-274.

To a solution of the above product (254 mg, 0.782 mmol), morpholine (150 mg, 1.72 mmol), and DMAP (102 mg, 0.831 mmol) in DCM (15 mL) was added N-[(dimethylamino)propyl]-N′-ethylcarbodiimide hydrochloride (190 mg, 0.991 mmol) at room temperature. The reaction mixture was stirred overnight. The reaction mixture was concentrated and the residue was dissolved in ethyl acetate and washed with 1 N aq. NaOH, 1 N aq. HCl, and water. The ethyl acetate layer was dried over sodium sulphate and concentrated to give the crude product, which was chromatographed on silica gel using EtOAc/MeOH to afford 138 mg (45%) of the product as a white solid. m.p.: 167-171; Mass 394[M+1].

¹HNMR (300 MHz; CDCl₃); δ: 3.5-3.9 (m, 8H, 4*CH₂); 6.94-7.5 (m, 8H, Ar.CH); 7.8 & 8.2 (brs, 2H, 2*NH); LCMS purity: 98%.

### Example 4

### Synthesis of 1-[1-(acetyl)piperidin-4-yl]-3-(adamant-1-yl)urea (Compound 2)

### Preparation of N-Acetyl piperid-4-yl amide

A reactor was charged with 1.00 mole-equivalent of 4-piperidinecarboxamide, 15.9 mole-equivalents of THF, and 1.23 mole-equivalents of N,N-(diisopropyl)ethylamine under a nitrogen atmosphere. The resulting mixture was cooled to 20° C. internal, and 1.10 mole-equivalents of acetic anhydride was added at such a rate as to maintain an internal temperature of less than 30° C. After addition was complete, the reaction mixture was stirred while maintaining an internal temperature of 20° C. The reaction contents were monitored until the amount of unreacted 4-piperidinecarboxamide was less than 1% relative to N-acetyl piperid-4-yl amide product (typically about 4-10 hours). The precipitated product was collected by filtration and washed with THF to remove excess (diisopropyl)ethylamine hydrochloride. The solid product was dried to constant weight in a vacuum oven under a nitrogen bleed while maintaining an internal temperature of ≦50° C. to afford the product as a white solid in 94% yield.

¹H NMR (CD₃OD) δ: 4.48-4.58 (bd, 1H), 3.92-4.01 (bd, 1H), 3.08-3.22 (m, 1H), 2.62-2.74 (m, 1H), 2.44-2.53 (m, 1H), 2.12 (s, 3H), 1.88-1.93 (m, 2H), 1.45-1.72 (m, 2H); MS: 171 [M+H]⁺; m.p. 172-174° C.

### Preparation of 1-(1-Acetylpiperidin-4-yl)-3-(adamant-1-yl)urea

A reactor was charged with 1.00 mole-equivalents of N-acetyl piperid-4-yl amide, 0.87 mole-equivalents of 1-adamantyl amine, and 49.7 mole-equivalents of acetonitrile, and the resulting mixture was heated to 75° C. internal under a nitrogen atmosphere. (Diacetoxyiodo)benzene (1.00 mole-equivalents) was charged portionwise in such a way that the reaction mixture was maintained between 75-80° C. internal. After the (diacetoxyiodo)benzene was added, the reaction mixture was heated to 80° C. internal. The reaction contents were monitored until the amount of unreacted 1-adamantyl amine was less than 5% relative to product N-(1-acetylpiperidin-4-yl)-N′-(adamant-1-yl)urea (typically about 1-6 hours). After completion, the reaction mixture was cooled to 25° C. internal, and approximately 24 mole-equivalents of solvent was distilled out under vacuum while maintaining internal temperature below 40° C. The reaction mixture was cooled with agitation to 0-5° C. internal and stirred for an additional 2 hours. The technical product was collected by filtration and washed with acetonitrile. The crude product was dried to constant weight in a vacuum oven under a nitrogen bleed maintaining an internal temperature of ≦50° C. The dried, crude product was slurried with water maintaining an internal temperature of 20±5° C. internal for 4 hours and then collected by filtration. The filter cake was washed with heptane under a nitrogen atmosphere then dried to constant weight in a vacuum oven under a nitrogen bleed maintaining an internal temperature of ≦70° C. to afford product as a white solid in 72% yield based on 1-adamantyl amine.

¹H NMR (DMSO-d₆) δ: 5.65-5.70 (bd, 1H), 5.41 (s, 1H), 4.02-4.10 (m, 1H), 3.61-3.70, (m, 1H), 3.46-3.58 (m, 1H), 3.04-3.23 (m, 1H), 2.70-2.78 (m, 1H), 1.98 (s, 3H), 1.84 (s, 6H), 1.64-1.82 (m, 2H), 1.59 (s, 6H), 1.13-1.25 (m, 1H), 1.00-1.12 (m, 1H); MS: 320 [M+H]⁺; m.p. 202-204° C.

### Example 5

### Synthesis of 1-[1-(methylsulfonyl)piperidin-4-yl]-3-(adamant-1-yl)urea (Compound 5)

### Preparation of N-Methanesulfonyl piperid-4-yl amide

A reactor was charged with 1.0 mole-equivalent of 4-piperidinecarboxamide,

16.4 mole-equivalents of THF, and 1.2 mole-equivalents of N,N-(diisopropyl)ethylamine under a nitrogen atmosphere. The resulting mixture was cooled to 0-5° C. internal, and 1.2 mole-equivalents of methanesulfonyl chloride was added at such a rate as to maintain an internal temperature of less than 10° C. After addition was complete, the reaction mixture was stirred allowing the temperature to rise to 20° C. internal. The reaction contents were monitored until the amount of unreacted 4-piperidinecarboxamide was less than 1% relative to N-methanesulfonyl piperid-4-yl amide product (typically about 2-12 hours). The precipitated product was collected by filtration then washed with dichloromethane to remove excess (diisopropyl)ethylamine hydrochloride. The solid product was dried to constant weight in a vacuum oven under a nitrogen bleed maintaining an internal temperature of ≦50° C. to afford product as a light yellow solid in 87% yield.

¹H NMR (DMSO-d₆) δ: 7.30 (s, 1H), 6.91 (s, 1H), 3.46-3.59 (m, 2H), 2.83 (s, 3H), 2.60-2.76 (m, 2H), 2.08-2.24 (m, 1H), 1.70-1.86 (m, 2H), 1.43-1.62 (m, 2H); MS: 207 [M+H]⁺; m.p. 126-128° C.

### Preparation of 1-(1-Methanesulfonyl piperidin-4-yl)-3-(adamant-1-yl)urea

A reactor was charged with 1.00 mole-equivalents of N-methanesulfonyl piperid-4-yl amide, 1.06 mole-equivalents of 1-adamantyl amine, and 39.3 mole-equivalents of acetonitrile, and the resulting mixture was heated to 40° C. internal under a nitrogen atmosphere. (Diacetoxyiodo)benzene (1.20 mole-equivalents) was charged portionwise in such a way that the reaction mixture was maintained below 75° C. internal. After the (diacetoxyiodo)benzene had been added, the reaction mixture was heated at 65-70° C. internal, and the reaction contents monitored until the amount of unreacted 1-adamantyl amine was less than 5% relative to product N-(1-methanesulfonyl piperidin-4-yl)-N′-(adamant-1-yl)urea (typically less than about 6 hours). The resulting mixture was cooled to 20° C. internal and filtered to remove a small amount of insoluble material. The filtrate was allowed to stand for 48 hours at which point the precipitated product was collected by filtration. The solid product was dried to constant weight in a vacuum oven under a nitrogen bleed maintaining an internal temperature of ≦50° C. to afford product in 58% yield based on N-methanesulfonyl piperid-4-yl amide.

¹H NMR (CDCl₃) δ: 3.95-4.08 (m, 2H), 3.74-3.82 (m, 2H), 3.63-3.82 (m, 1H), 3.78 (s, 3H), 3.70-3.80 (m, 2H), 2.02-2.12 (m, 5H), 1.90 (s, 6H), 1.67 (s, 6H), 1.40-1.50 (m, 2H); MS: 356 [M+H]⁺; m.p. 228-229° C.

### Percent Inhibition

The percent inhibition for each of compounds 1-5 was determined according to the following procedure:

The substrate for the reaction was:

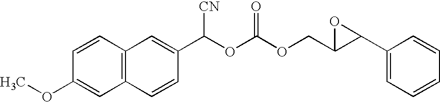

Cyano(2-methoxynaphthalen-6-yl)methyl (3-phenyloxiran-2-yl)methyl carbonate (CMNPC; Jones P. D. et. al.; Analytical Biochemistry 2005; 343: pp. 66-75)

A standard 96 well plate has rows typically identified by letter and columns identified by numbers. Therefore, well A2 would refer to a well in the first row and second column of the plate.

In a black 96 well plate, all the wells are filled with 150 μL of buffer A (Buffer A: Bis/Tris HCl, 25 mM, pH 7.0 plus 0.1 mg/mL BSA). DMSO (2 microliters) was added in well A2 and A3, and then was added 2 μL of inhibitor solution in A1 and A4 through A12. 150 μL of buffer A was added to row A, then mixed several times and 150 μL of the solution was transferred to row B. This mixing and transfer was repeated up to row H. 20 μL of buffer A was added in column 1 and 2, then 20 μL of enzyme solution was added to columns 3 through 12. The plate was incubated for 5 minutes in the plate reader at 30° C. During incubation, the working solution of substrate was prepared by mixing 3.68 mL of buffer A with 266 μL of 0.5 mM substrate solution. At t=0, 30 μL of working substrate solution was added and readings were started ([S]_{final}: 5 μM). The readings were done at ex: 330 nm (bandwidth 20 nm) and em: 465 nm (bandwidth 20 nm) every 30 second for ten minutes using a fluorescent plate reader (Spectromax M5, Molecular Devices).

Table 3 shows percent inhibition of Compounds 1-5 (as referred to in Table 2) when tested at 50 nM.

TABLE 3

| | % Inhibition at |
|---|---|
| Compound | 50 nM |
| 1 | 82 |
| 2 | 89 |
| 3 | 81 |
| 4 | 85 |
| 5 | 94 |

### Example 6

### Metabolic Syndrome Model 1

A diet induced obesity mouse model was used to evaluate the efficacy of the sEH inhibitor 1-[1-(methylsulfonyl)piperidin-4-yl]-3-(adamant-1-yl)urea (compound 5) for the treatment of metabolic syndrome and the adverse conditions related thereto.

The study was performed using 7-8 week old male C57B1/6 mice. The mice were acclimated a minimum of five days prior to the start of study and were housed five per cage in microisolators in a 12:12 light/dark cycle (all work was done in a BioBubble Hood™). Water and food was provided ad libitum.

The mice were provided a high-fat, high-fructose diet for a total of 11 weeks, within the first 5 weeks the animals became obese, insulin resistant, have increased plasma cholesterol and mildly hypertensive. The mice were separated into three groups, each group consisting of 10 mice after the first 5 weeks on the high-fat, high-fructose diet. The mice continued to receive the high-fat, high-fructose diet but also began to receive treatment for the remaining 6 weeks of the study for a total of 11 weeks of study. During the treatment phase, Group 1 was administered vehicle alone, perorally, twice a day for six weeks (control group); group 2 was administered 20 mg/kg of compound 5, perorally, twice a day for six weeks; group 3 was administered 60 mg/kg compound 5, perorally, twice a day for six weeks. At intervals of 3 and 5.5 weeks after the beginning of the treatment phase of the study, glucose tolerance tests were administered. At the beginning of the treatment phase and after 5 weeks of dosing, samples were collected for plasma cholesterol measurements. Blood pressure was measured 3 weeks after the start of the treatment phase of the study.

### Results

### Obesity

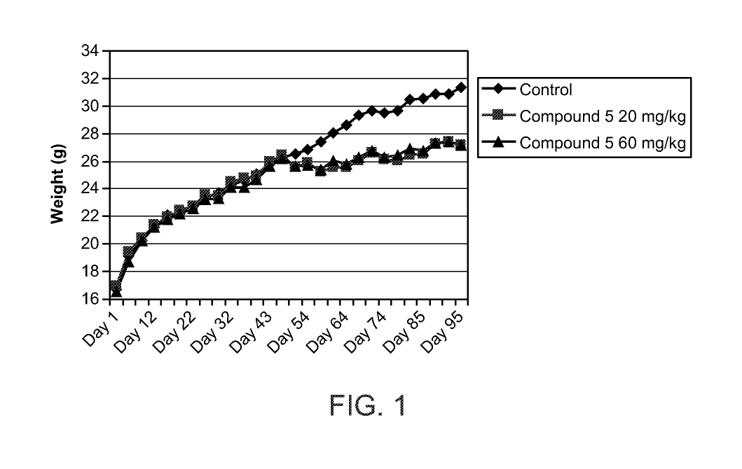

Those mice treated with either dose of compound 5 exhibited a stabilization in body weight as compared to the vehicle control group which continues to gain weight (FIG. 1). This stabilization in body weight begins with the initiation of dosing of compound 5.

### Glucose Tolerance

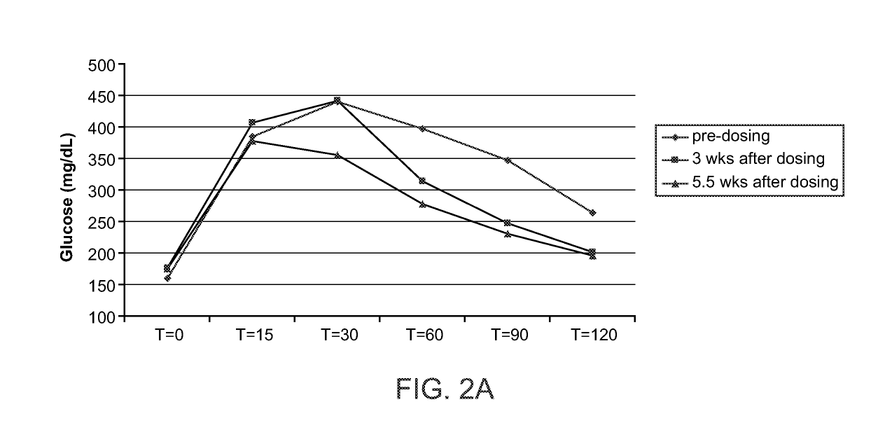

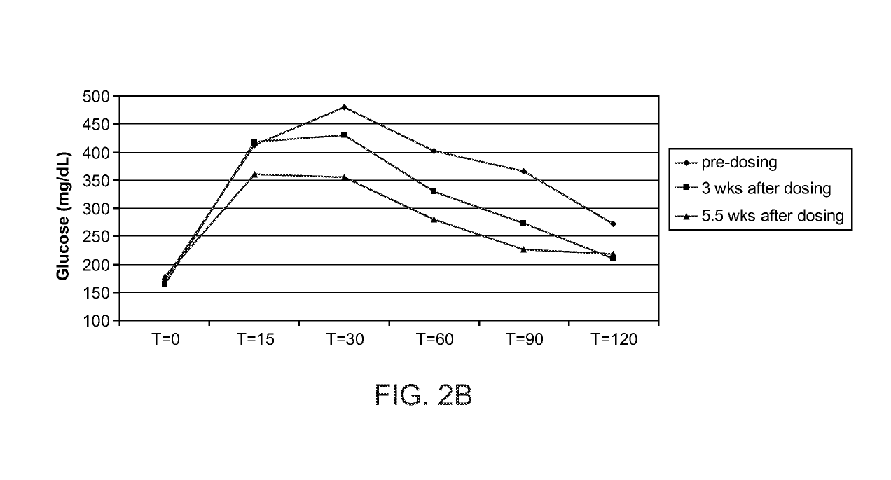

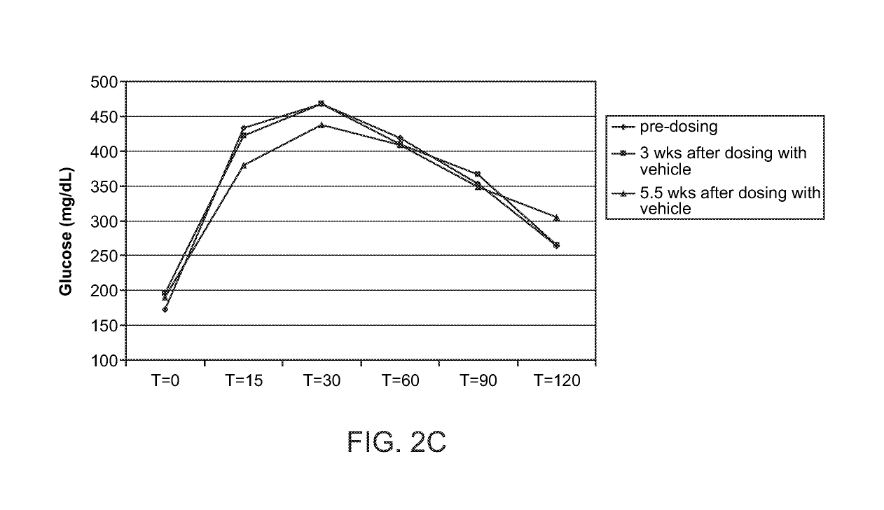

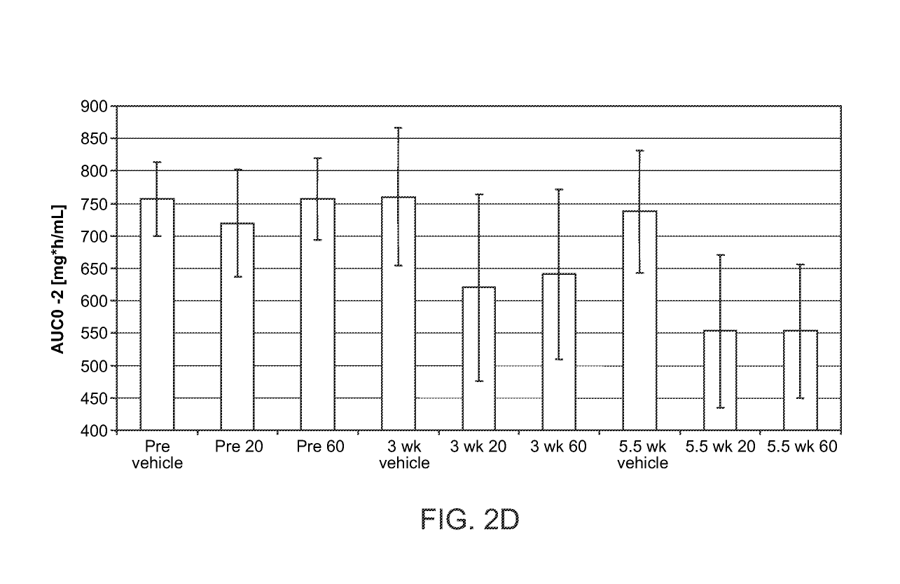

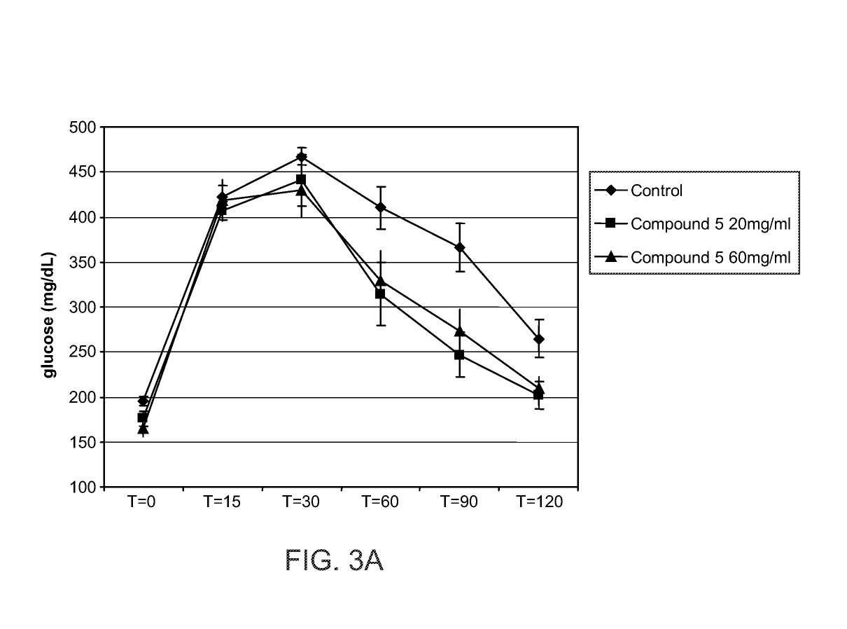

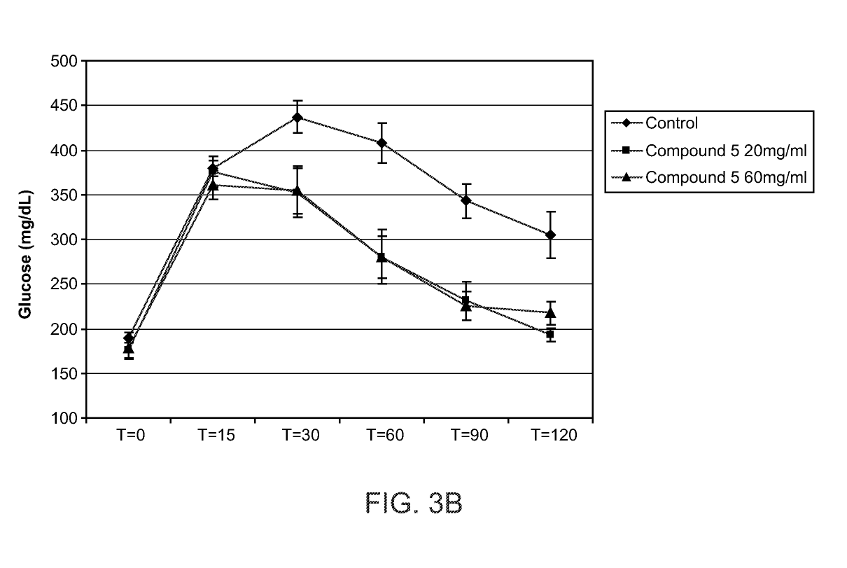

FIGS. 2A-C and 3A-B show the amount of glucose measured in mg/dL in a serum sample taken from the mice subjects at 0, 15, 30, 60, 90, and 120 minutes. FIG. 2A shows data obtained from mice administered with 20 mg/kg of compound 5 with serum samples taken pre-dosing, 3 weeks after administration of the compound, and 5.5 weeks after administration of the compound, or 8 or 10.5 weeks after the initiation of the high-fat, high-fructose diet. FIG. 2B shows data obtained from mice administered with 60 mg/kg of compound 5 with serum samples taken pre-dosing, 3 weeks and 5.5 weeks after administration of the compound, or 8 or 10.5 weeks after the initiation of the high-fat, high-fructose diet. FIG. 2C shows data obtained from mice administered with vehicle alone (control group), with serum samples taken pre-dosing, 3 weeks and 5.5 weeks after administration of the vehicle, or 8 or 10.5 weeks after the initiation of the high-fat, high-fructose diet. In FIG. 2D the area under the curve (AUC) for the data between time 0 to 120 minutes was calculated for all the GTT data. The AUC was calculated using a linear trapezoidal sum of the area from time 0 to 120 minutes after dosing of glucose. This method of depicting the GTT results allows for a quantitative comparison of all of the groups at the different time points at which the GTT was performed. FIGS. 3A and 3B show plasma glucose level data obtained from mice administered with either 20 mg/kg of compound 5, 60 mg/kg compound 5, or vehicle alone orally twice daily, at 8 weeks after the initiation of the high-fat, high-fructose diet (FIG. 3A) and 10.5 weeks (FIG. 3B) after the initiation of the high-fat, high-fructose diet or 3 weeks after administration of the compound, and 5.5 weeks after administration of the compound.

As indicated in these Figures, the mice treated with compound 5 exhibited a decrease in serum glucose relative to the control group as determined by an GTT (interperitonial glucose tolerance test) test. This result indicates that mice treated with compound 5 have improved glucose handling resulting in a decrease in glucose intolerance. The animals receiving both 20 and 60 mg/kg of compound 5 twice a day had statistically lower area under the curve for plasma glucose after an interperitonial injection of glucose compared to the vehicle treated animals and compared to the values at the start of treatment (p<0.01). The decrease is plasma glucose in both mice groups receiving compound 5 is detectable as early as 3 weeks after initiation of administration of the compound (FIGS. 2A-2D) at 8 weeks after the initiation of the high-fat, high-fructose diet and 3 weeks after initiation of the administration of the compound (FIG. 3A) and 10.5 weeks after the initiation of the high-fat, high-fructose diet and 5.5 weeks after initiation of the administration of the compound (the last time point collected in this assay) (FIG. 3B). This decrease in plasma glucose reflects a therapeutic improvement in glucose handling with treatment by compound 5.

### Blood Pressure

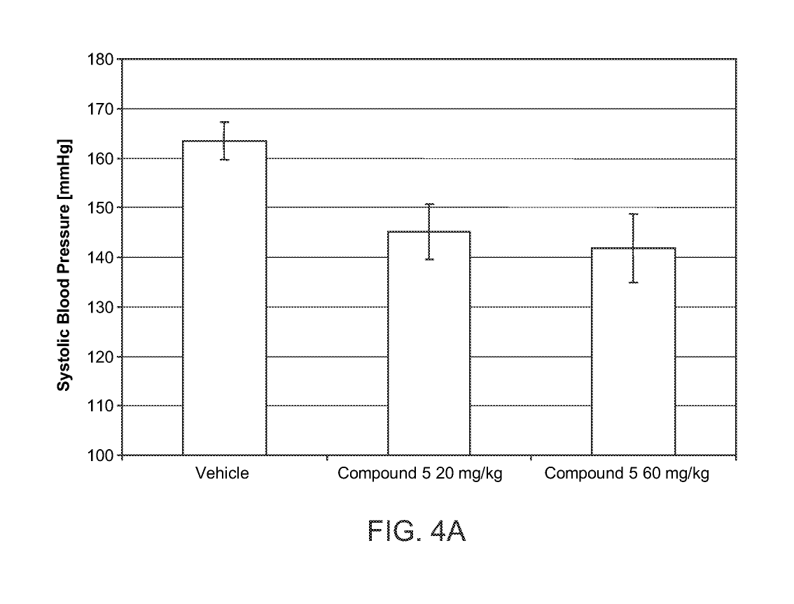

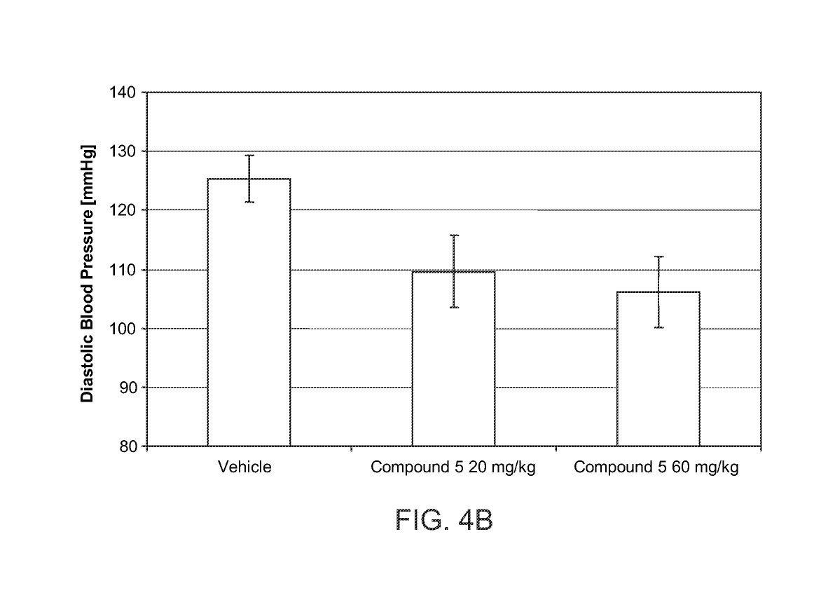

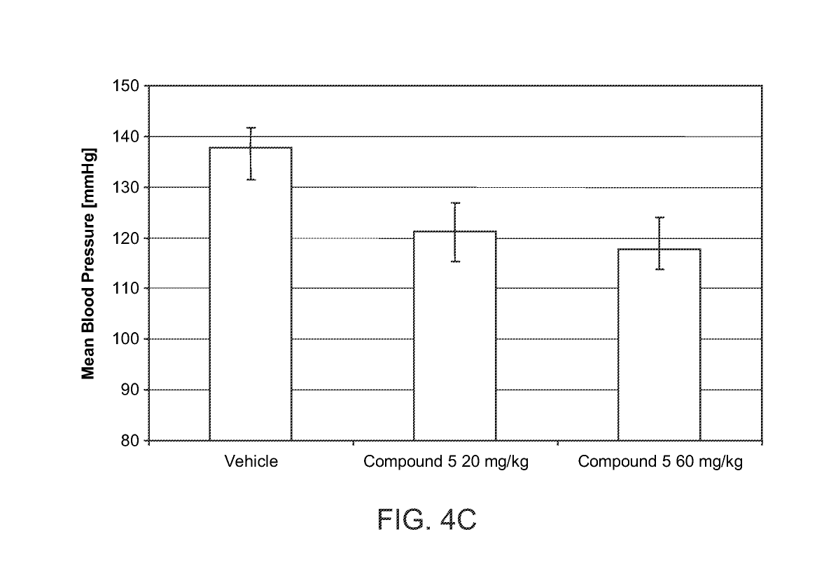

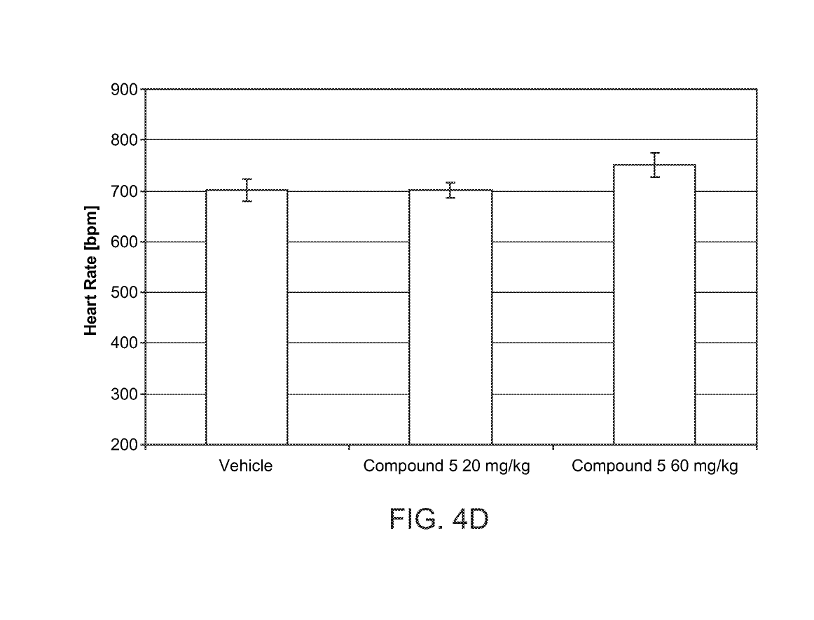

The systolic and diastolic blood pressure (measured in mm Hg) in the vehicle group are elevated relative to normal blood pressure in C57B1/6 mice after 8 weeks of high-fat and high-fructose diet. Both systolic and diastolic blood pressure was reduced in the mice treated with compound 5 as compared to the control group (FIGS. 4A and 4B). The animals receiving 60 mg/kg twice a day had statistically significantly lower blood pressure relative to the vehicle treated group (p<0.05). The mean blood pressure of the mice treated with compound 5 was similarly reduced as compared to the control group (FIG. 4C). The heart rate of the mice in all three groups was within the same range (FIG. 4D). Therefore, compound 5 did not significantly alter heart rate.

### Cholesterol Levels

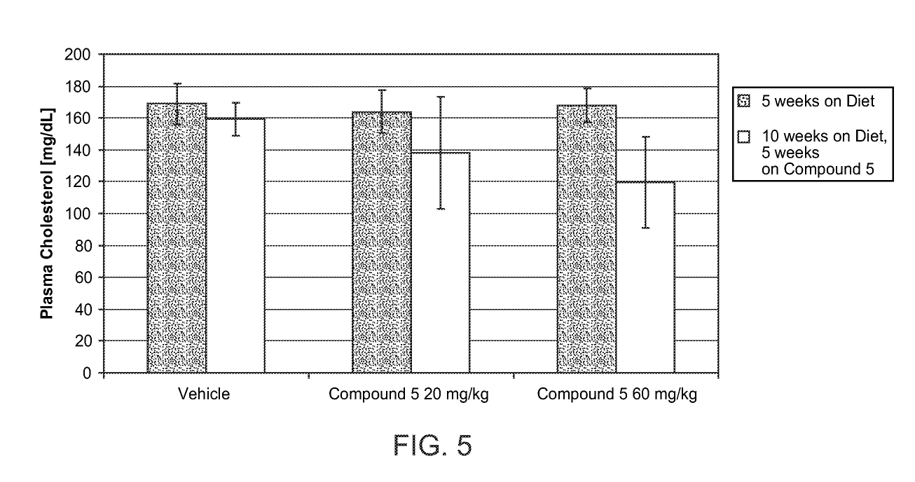

The plasma cholesterol levels were reduced in mice treated with compound 5 relative to the control group (FIG. 5). The animals receiving 60 mg/kg twice a day had statistically lower cholesterol level relative to the vehicle treated animals (p<0.01)

### Conclusions

Taken together, the above results indicate that sEH inhibitors of the present inventions, specifically the compound 1-[1-(methylsulfonyl)piperidin-4-yl]-3-(adamant-1-yl)urea, are useful in treating metabolic syndrome and reducing the adverse conditions associated with this syndrome such as obesity, glucose intolerance, high blood pressure, and elevated serum cholesterol.

Additional details of the study are provided below.

### Experimental Diets

Diet is defined as both solid food and liquid.

HF diet with 45% fat by calorie (Research Diets, D12451)

High-Fructose (degassed 7up)

Experimental groups: n=10/group

Group 1) High-Fat Diet

Group 2) High-Fat Diet+compound 5-20 mg/kg p.o. (perorally) b.i.d. (twice a day)

Group 3) High-Fat Diet+compound 5-60 mg/kg p.o. (perorally) b.i.d. (twice a day)

Test Procedures

Throughout duration of the study body weight, food, and fructose consumption was measured twice a week. Blood pressure and heart rate was measured one week.

Assay Protocol

### Day 0:

Mice were randomized on the basis of average Body Weight in each of the three groups (n=10/gp).

Diet begins.

### Day 28:

GTT—Animals were fasted for 4 hours followed by a glucose load (2 g/kG body wt). The tip (3 mm) of the tail was excised and blood samples were taken for glucose clearance measurements (Glucometer) at T=0, 15, 30, 60, 90, and 120 minutes. This test determines if the animal is has glucose intolerance.

### Day 34:

Plasma was analyzed using a lipid and chem panel analysis (Lipid—total cholesterol, HDL, LDL and Triglycerides, FFA).

### Day 35:

p.o. (perorally) dosing of compound 5 at 20 mg/kg and at 60 mg/kg begins b.i.d. twice a day) for 5 weeks

### Day 56:

Measure blood pressure using a non-invasive CODA 6 occlusion cuff system

### Example 6

### Metabolic Syndrome Model 2

A diet induced obesity mouse model was used to evaluate the efficacy of three sEH inhibitors; 1-[1-(acetyl)piperidin-4-yl]-3-(4-trifluoromethylphenyl)urea (compound 3); 1-[1-(methylsulfonyl)piperidin-4-yl]-3-(4-trifluoromethylphenyl)urea (compound 4) and 1-[1-(methylsulfonyl)piperidin-4-yl]-3-(adamant-1-yl)urea (compound 5) for the treatment of metabolic syndrome and the adverse conditions related thereto.

Seven groups of 10 wild-type mice were entered onto the study. Five groups were placed on an ad libitum high-fat, high-fructose diet (HF); two groups were fed ad libitum with standard rodent chow and water (NC). Animals were maintained on the respective diet for the entire 12 weeks of the study. Beginning in Week 8 and continuing for the rest of the in-life period, mice were dosed twice daily by oral gavage with vehicle (CMC-Tween), 10 mg/kg/day in drinking water of Losartan or with 60 mg/kg of compound 3, compound 4 or compound 5. Losartan (Cozaar®) is a compound approved by the FDA for the treatment of hypertension, reducing the risk of stroke in a patient with hypertension and left ventricular hypertrophy, and treatment of diabetic nephropathy with an elevated serum creatine and proteinuria in patients with type 2 diabetes and a history of hypertension.

Glucose tolerance tests (GTTs) were performed in Week 7 (prior to the start of dosing) and Week 12 (after 4 weeks of dosing). Body weights were recorded semi-weekly throughout the study whereas chow consumption and liquid intake were recorded weekly. Plasma was collected from untreated (Week 7, prior to the start of dosing) and treated (at the end of in-life) animals and submitted for the determination of plasma cholesterol. At the end of the in-life period, animals were sacrificed after terminal bleeds and discarded; no necropsies were performed.

### Results

s-EH inhibitors were well-tolerated. Among the animals on the NC, body weights were similar whether dosed with vehicle or compound 5. Neither food consumption, liquid intake, nor total caloric intake was apparently altered following the start of dosing with vehicle or test compounds.

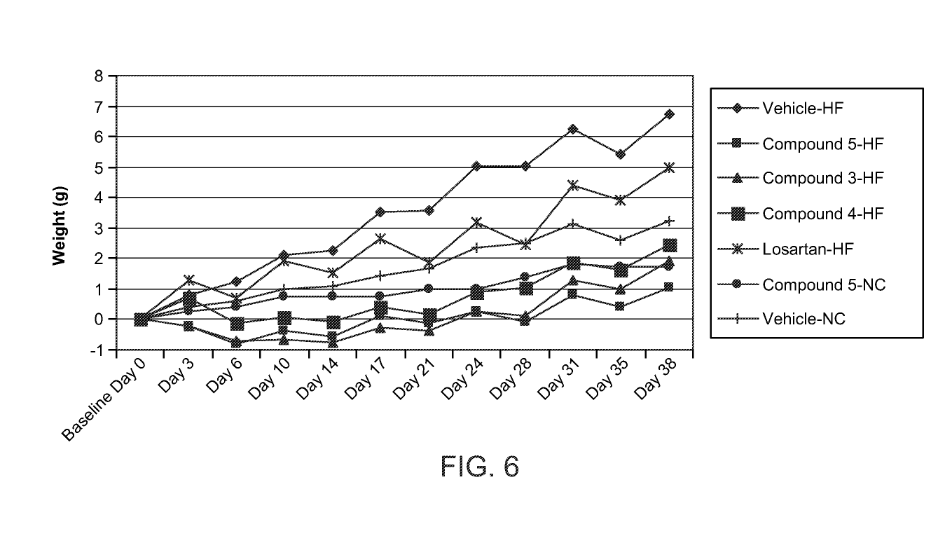

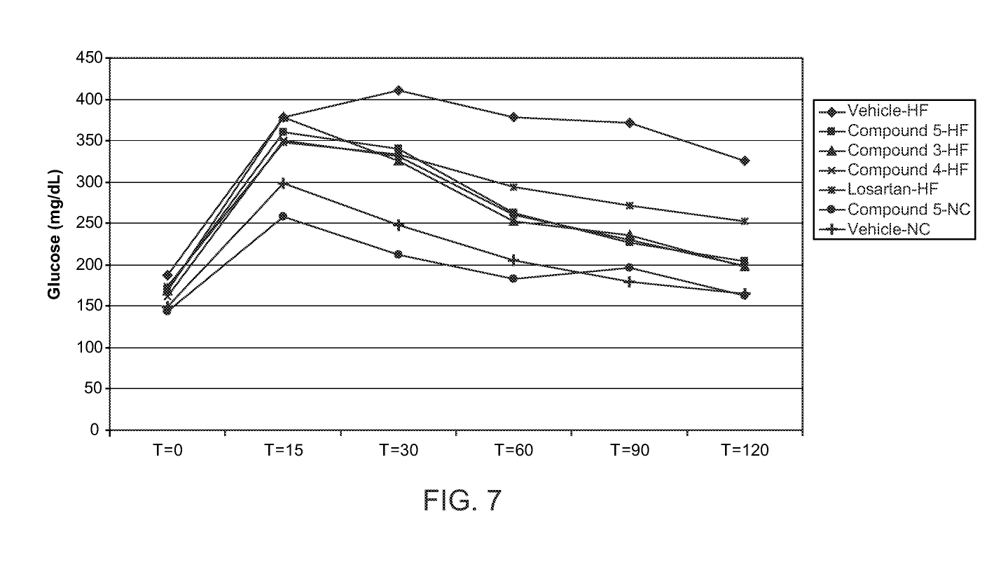

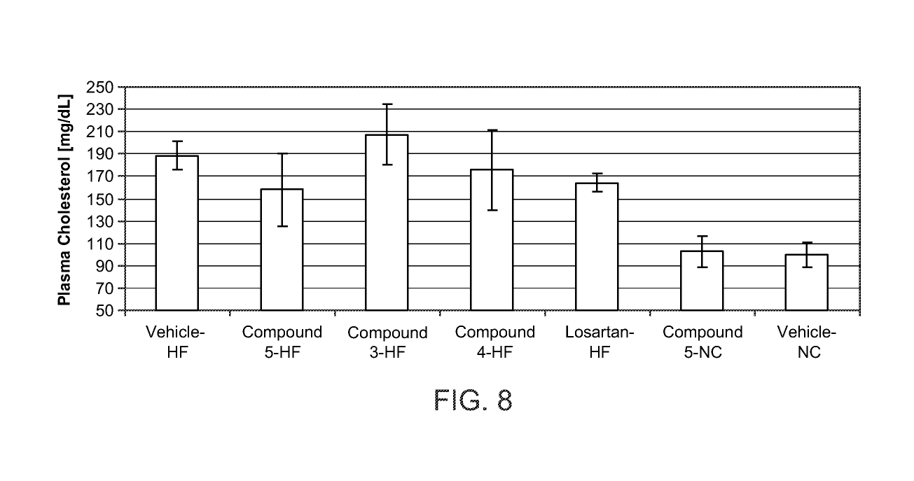

Among HF-fed animals, those dosed with compound 3, compound 4, compound 5 or Losartan gained less weight during the study than those receiving vehicle (FIG. 6). However, the attenuated weight gain was statistically significant with compound 3 and compound 5, achieving statistical significance during the second week of dose administration. HF-fed mice that were dosed with compound 3, compound 4 or compound 5 demonstrated statistically significant improvements in glucose tolerance compared to animals receiving vehicle, although the glucose tolerance still did not match that of NC-fed mice (FIG. 7). Similarly, dosing of HF mice with compound 5 resulted in statistically significant decreases in cholesterol, but levels were still higher than those in NC-fed mice (FIG. 8).

### CONCLUSIONS

Taken together, the above results indicate that sEH inhibitors of the present inventions, specifically the compounds 1-[1-(acetyl)piperidin-4-yl]-3-(4-trifluoromethylphenyl)urea (compound 3); 1-[1-(methylsulfonyl)piperidin-4-yl]-3-(4-trifluoromethylphenyl)urea (compound 4) and 1-[1-(methylsulfonyl)piperidin-4-yl]-3-(adamant-1-yl)urea (compound 5) are useful in treating metabolic syndrome and improving all or some of the adverse conditions associated with this syndrome such as elevated weight gain, poor glucose tolerance, and increased plasma cholesterol. Additionally, while plasma cholesterol is improved by the sEH inhibitors of the present inventions, it is contemplated that these compounds are also useful for improving low-density lipoproteins (LDL) levels and/or high-density lipoproteins (HDL) levels.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A method for inhibiting the onset of metabolic syndrome in a mammalian subject predisposed thereto, which method comprises administering to the subject an effective amount of a sEH inhibitor, wherein the sEH inhibitor is a compound of Formula (II) or a pharmaceutically acceptable salt thereof: 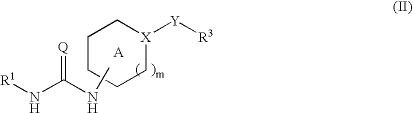
wherein:
Q is selected from the group consisting of O and S;
R¹ is selected from the group consisting of aryl, substituted aryl, heteroaryl, substituted heteroraryl, cycloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂;
R³ is selected from the group consisting of alkyl, substituted alkyl, or heterocycloalkyl; and
m is selected from the group consisting of zero, 1, and 2.

2. The method of claim 1, wherein the sEH inhibitor is a compound of Formula (IIa) or a pharmaceutically acceptable salt thereof: 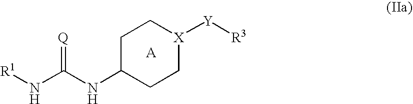
wherein:
Q is selected from the group consisting of O and S;
R¹ is selected from the group consisting of aryl, substituted aryl, heteroaryl, substituted heteroraryl, cycloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂; and
R³ is selected from the group consisting of alkyl, substituted alkyl, or heterocycloalkyl.

3. The method of claim 2, wherein the compound is selected from the group consisting of 1-[1-(methylsulfonyl)piperidin-4-yl]-3-(adamant-1-yl)urea, 1-[1-(acetyl)piperidin-4-yl]-3-(adamant-1-yl)urea, 1-[1-(acetyl)piperidin-4-yl]-3-(4-trifluoromethylphenyl)urea, 1-[1-(methylsulfonyl)piperidin-4-yl]-3-(4-trifluoromethylphenyl)urea and 1-[3-(morpholino-4-carbonyl)phenyl]-3-(4-trifluoromethylphenyl)urea.

4. The method of claim 3, wherein the compound is 1-[1-(methylsulfonyl)piperidin-4-yl]-3-(adamant-1-yl)urea.

5. The method of claim 3, wherein the compound is 1-[1-(acetyl)piperidin-4-yl]-3-(4-trifluoromethylphenyl)urea.

6. The method of claim 3, wherein the compound is 1-[1-(methylsulfonyl)piperidin-4-yl]-3-(4-trifluoromethylphenyl)urea.

7. A method for treating one or more conditions associated with metabolic syndrome in a subject, comprising administering to the subject an effective amount of a sEH inhibitor, wherein the sEH inhibitor is a compound of Formula (II) or a pharmaceutically acceptable salt thereof: 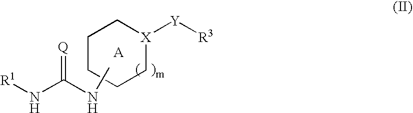
wherein:
Q is selected from the group consisting of O and S;
R¹ is selected from the group consisting of aryl, substituted aryl, heteroaryl, substituted heteroraryl, cycloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂;
R³ is selected from the group consisting of alkyl, substituted alkyl, or heterocycloalkyl; and
m is selected from the group consisting of zero, 1, and 2,
wherein the conditions are selected from the group consisting of incipient diabetes, obesity, glucose intolerance, high blood pressure, elevated serum cholesterol, and elevated triglycerides.

8. The method of claim 7, wherein the sEH inhibitor is a compound of Formula (IIa) or a pharmaceutically acceptable salt thereof: 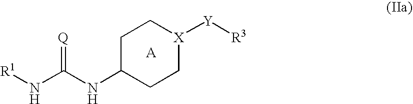
wherein:
Q is selected from the group consisting of O and S;
R¹ is selected from the group consisting of aryl, substituted aryl, heteroaryl, substituted heteroraryl, cycloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂; and
R³ is selected from the group consisting of alkyl, substituted alkyl, or heterocycloalkyl.

9. The method of claim 8, wherein the compound is selected from the group consisting of 1-[1-(methylsulfonyl)piperidin-4-yl]-3-(adamant-1-yl)urea, 1-[1-(acetyl)piperidin-4-yl]-3-(adamant-1-yl)urea, 1-[1-(acetyl)piperidin-4-yl]-3-(4-trifluoromethylphenyl)urea, 1-[1-(methylsulfonyl)piperidin-4-yl]-3-(4-trifluoromethylphenyl)urea and 1-[3-(morpholino-4-carbonyl)phenyl]-3-(4-trifluoromethylphenyl)urea.

10. The method of claim 9, wherein the compound is 1-[1-(methylsulfonyl)piperidin-4-yl]-3-(adamant-1-yl)urea.

11. The method of claim 9, wherein the compound is 1-[1-(acetyl)piperidin-4-yl]-3-(4-trifluoromethylphenyl)urea.

12. The method of claim 9, wherein the compound is 1-[1-(methylsulfonyl)piperidin-4-yl]-3-(4-trifluoromethylphenyl)urea.

13. The method of any one of claims 7-12, wherein two or more of the conditions are treated by administering the sEH inhibitor compound.

14. A method for treating a metabolic condition in a subject, comprising administering to the subject an effective amount of a sEH inhibitor, wherein the sEH inhibitor is a compound of Formula (II) or a pharmaceutically acceptable salt thereof: 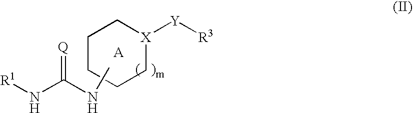
wherein:
Q is selected from the group consisting of O and S;
R¹ is selected from the group consisting of aryl, substituted aryl, heteroaryl, substituted heteroraryl, cycloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂;
R³ is selected from the group consisting of alkyl, substituted alkyl, or heterocycloalkyl; and
m is selected from the group consisting of zero, 1, and 2,
wherein the metabolic condition is selected from the group consisting of conditions comprising obesity, glucose intolerance, high blood pressure, elevated serum cholesterol, and elevated triglycerides, and combinations thereof

15. The method of claim 14, wherein the sEH inhibitor is a compound of Formula (IIa) or a pharmaceutically acceptable salt thereof: 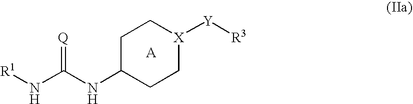
wherein:
Q is selected from the group consisting of O and S;
R¹ is selected from the group consisting of aryl, substituted aryl, heteroaryl, substituted heteroraryl, cycloalkyl, substituted cycloalkyl, heterocycloalkyl, and substituted heterocycloalkyl;
X is C or N; provided that when X is C then ring A is phenyl and when X is N then ring A is piperidinyl;
Y is selected from the group consisting of CO and SO₂; and
R³ is selected from the group consisting of alkyl, substituted alkyl, or heterocycloalkyl.

16. The method of claim 15, wherein the compound is selected from the group consisting of 1-[1-(methylsulfonyl)piperidin-4-yl]-3-(adamant-1-yl)urea, 1-[1-(acetyl)piperidin-4-yl]-3-(adamant-1-yl)urea, 1-[1-(acetyl)piperidin-4-yl]-3-(4-trifluoromethylphenyl)urea, 1-[1-(methylsulfonyl)piperidin-4-yl]-3-(4-trifluoromethylphenyl)urea and 1-[3-(morpholino-4-carbonyl)phenyl]-3-(4-trifluoromethylphenyl)urea.

17. The method of claim 16, wherein the compound is 1-[1-(methylsulfonyl)piperidin-4-yl]-3-(adamant-1-yl)urea.

18. The method of claim 16, wherein the compound is 1-[1-(acetyl)piperidin-4-yl]-3-(4-trifluoromethylphenyl)urea.

19. The method of claim 16, wherein the compound is 1-[1-(methylsulfonyl)piperidin-4-yl]-3-(4-trifluoromethylphenyl)urea.

20. The method of any one of claims 14-19, wherein the metabolic condition comprises obesity.

21. The method of any one of claims 14-19, wherein the metabolic condition comprises glucose intolerance.

22. The method of any one of claims 14-19, wherein the metabolic condition comprises high blood pressure.

23. The method of any one of claims 14-19, wherein the metabolic condition comprises elevated serum cholesterol.
